(19) United States
(12) Patent Application Publication
(10) Pub. No.: US 2024/0304335 A1
(43) Pub. Date: Sep. 12, 2024

(54) METHOD, SOFTWARE, AND SYSTEMS FOR PREDICTING RELAPSE OF PROSTATE CANCER TREATED BY RADIATION THERAPY

(71) Applicant: Purdue Research Foundation, West Lafayette, IN (US)

(72) Inventors: Guillermo Lorenzo Gomez, Coruna (ES); Hector Gomez Diaz, West Lafayette, IN (US); Victor Manuel Perez-Garcia, Ciudad Real (ES); Alessandro Reali, Pavia (IT)

(21) Appl. No.: 18/598,558

(22) Filed: Mar. 7, 2024

Related U.S. Application Data
(60) Provisional application No. 63/488,900, filed on Mar. 7, 2023.

(51) Int. Cl. G16H 50/30

(52) U.S. Cl. CPC G16H 50/30

(57) ABSTRACT

Methods of predicting relapse of a prostate cancer patient treated by radiation therapy, associated software, and systems for implementing such methods. Patient-specific PSA data are collected including nₚ measurements of serum prostate specific antigen (PSA) at nₚ dates for a newly-diagnosed prostate cancer patient before and after an external beam radiation therapy (EBRT) regimen. Patient-specific parameters P₀, ρₙ, ρₛ, ρ_{d}, and R_{D} are identified that render an optimal fit of the patient-specific PSA data to a personalized model. Using the personalized model and the patient-specific parameters, a set of one or more predicted PSA values are calculated at one or more time horizons from 0 to 2 years after the last PSA measurement. If and/or when a relapse of the prostate cancer patient will occur is predicted based on the one or more predicted PSA values.

### CROSS-REFERENCE TO RELATED APPLICATIONS

This application claims the benefit of provisional U.S. Patent Application No. 63/488,900 filed Mar. 7, 2023, the contents of which are incorporated herein by reference.

### STATEMENT REGARDING PRIOR DISCLOSURES BY AN INVENTOR OR JOINT INVENTOR

One or more of the inventors have made disclosures related to the general field of modeling and/or predicting prostate cancer progression and biochemical relapse following external beam radiation therapy in the following publications:
[1] Hughes et al. U.S. Patent Application Publication No. 2019/0198177 A1.
[2] Lorenzo G, Pérez-García V M, Mariño A, Pérez-Romasanta L A, Reali A, Gomez H., 2019, “Mechanistic modelling of prostate-specific antigen dynamics shows potential for personalized prediction of radiation therapy outcome,” J. R. Soc. Interface 16: 20190195. http://dx.doi.org/10.1098/rsif.2019.0195, hereinafter, referred to as “Lorenzo et al. (2019)”.
[3] G. Lorenzo, N. di Muzio, C. L. Deantoni, C. Cozzarini, A. Fodor, A. Briganti, F. Montorsi, V. M. Pérez-García, H. Gomez, A. Reali, “Patient-specific forecasting of post-radiotherapy prostate-specific antigen kinetics enables early prediction of biochemical relapse,” published Mar. 7, 2022, medRxiv 2022.03.07.22271524; https://doi.org/10.1101/2022.03.07.22271524, a copy of which is attached hereto in Appendix A.

### BACKGROUND OF THE INVENTION

The invention generally relates to medical assessments relating to conditions and treatments of patients. The invention particularly relates to methods, software, and systems for predicting relapse of prostate cancer patients treated by radiation therapy.

External beam radiotherapy (EBRT, or simply “radiotherapy”) is a standard treatment for prostate cancer (PCa) that is potentially available for patients of all ages to treat tumors ranging from low to high and very high risk. During EBRT, the prostate is exposed to an external source of radiation, which aims at disrupting the DNA in tumor cells' nuclei. The accumulation of radiation-induced damage along with the multiple genetic alterations underlying the development of PCa ultimately forces tumor cells to undergo programmed cell death. EBRT is usually delivered as daily fractions of approximately 2 to 3 Gy until completing a total dose ranging from 60 to 80 Gy. The use of daily doses higher than 2 Gy in the recent decades has led to so-called moderate hypofractionation. This treatment modality requires a lower number of radiation sessions, which presents pharmacoeconomic advantages. The efficacy of EBRT can be improved through combination with neoadjuvant or adjuvant androgen deprivation therapy (ADT). However, since ADT can produce several side effects, it is usually prescribed for intermediate and high-risk PCa patients only (i.e., those who present a higher risk of metastasis).

After the completion of EBRT, PCa progression in a patient is monitored by monitoring the serum levels of prostate-specific antigen (PSA), which is a standard clinical biomarker of PCa. The rationale for using PSA in post-EBRT patient follow-up is that blood levels of PSA tend to rise due to PCa growth. Thus, if the treatment is successful, radiation-induced tumor cell death should decrease PSA values to a minimum, which may vary from patient to patient and with prostate size. Otherwise, if EBRT does not eradicate the tumor completely, the surviving cancerous cells will ultimately drive tumor growth after EBRT conclusion and, hence, produce an increasing trend in PSA levels. This phenomenon is termed biochemical relapse and it is thus indicative of tumor recurrence. Studies show that approximately 20% to 50% of PCa patients undergoing radiotherapy as primary curative treatment will ultimately develop biochemical relapse within 5 to 10 years after treatment conclusion, respectively. Following the detection of biochemical relapse, tumor recurrence can be confirmed through biopsy and imaging methods, such as magnetic resonance imaging (MRI) and prostate-specific membrane antigen (PSMA) or choline positron emission tomography/computed tomography (choline PET/CT). To treat post-EBRT PCa recurrence, there are several therapeutic strategies that depend on whether the recurrence is local or metastatic.

Serum PSA may exhibit natural fluctuations (e.g., due to diet and lifestyle), a smooth increase caused by benign prostatic enlargement (i.e., benign prostatic hyperplasia), and transient peaks due to ADT termination or the so-called PSA bounce, which consists of a temporary PSA increase of 0.1 to 0.5 ng/mL usually occurring within the first 24 months after EBRT conclusion. These phenomena may hamper the detection of biochemical relapse following EBRT. Thus, the current clinical criteria to identify a biochemical relapse typically require PSA to exhibit a consistent rising trend over time. For example, a standard criterion with widespread use in current clinical practice identifies a biochemical relapse as a PSA increase larger than 2 ng/mL over the detected PSA nadir (i.e., the minimum PSA value measured for a patient). Additionally, multiple studies have been devoted to analyzing PSA dynamics after EBRT and its correlation with the pathological features of tumor recurrence to define other PSA-based markers that improve the identification and prognostic assessment of biochemical relapse and PCa recurrence. For instance, the detection of a rapid decline of PSA right after treatment, overall high PSA values during post-treatment monitoring, an early nadir, a high value of the nadir, and a low PSA doubling time during biochemical relapse (i.e., the time it would take PSA to exhibit a two-fold increase) have been correlated with a poorer prognosis, including metastatic disease and lower patient survival. Alternatively, post-EBRT PSA dynamics has also been analyzed by fitting empirical equations to patient-specific longitudinal series of PSA values. In particular, successful results have been reported by leveraging a biexponential formula, which uses the sum of two terms: an exponential decay to capture the usual post-treatment decline in PSA observed in all patients, and a rising exponential to represent biochemical relapse, which vanishes when this empirical model is fit to data from cured patients.

However, the current criteria of biochemical relapse and the majority of PSA-based markers only enable assessment of this event upon its direct observation. Hence, these approaches may ultimately delay the diagnosis and treatment of tumor recurrence, thereby potentially reducing the chances of successfully controlling the disease. Additionally, observational metrics and models of PSA dynamics offer a limited representation of the underlying tumor dynamics that ultimately regulate the observed changes of PSA in each patient.

Several studies have investigated mechanistic models of PCa growth and PSA dynamics in various scenarios, including untreated tumor growth, hormone therapy, cytotoxic and antiangiogenic therapies, and after radical prostatectomy. Since radiotherapy is used for the treatment of many types of cancer, the study of tumor response to radiation and the forecasting of patient-specific radiotherapeutic outcomes using mechanistic models constitute a rich area of research. Nevertheless, there is a dearth of mechanistic models providing a coupled description of tumor and PSA dynamics following radiotherapy.

Therefore, it would be desirable to have a method and related systems and/or apparatus that would provide a way to identify tumor recurrence earlier after external radiotherapy than possible with conventional methods so as to be able to anticipate secondary treatments and maximize the chances of tumor control.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, methods, software, and systems for predicting relapse of prostate cancer in patients treated by radiation therapy.

According to one nonlimiting aspect, a method of predicting relapse of a prostate cancer patient treated by radiation therapy is provided. The method includes collecting patient-specific PSA data comprising nₚ measurements of serum Prostate Specific Antigen (PSA) at nₚ dates for a newly-diagnosed prostate cancer patient before and after an external beam radiation therapy (EBRT) regimen, identifying patient-specific parameters P₀, ρₙ, ρₛ, ρ_{d}, and R_{D} that render an optimal fit of the patient-specific PSA data to a personalized model; calculating, using the personalized model and the patient-specific parameters, a set of one or more predicted PSA values at one or more time horizons from 0 to 2 years after the last PSA measurement, and predicting if and/or when a relapse of the prostate cancer patient will occur based on the one or more predicted PSA values. Optionally, the model parameters and ensuing predictions may be updated by repeating the steps of collecting patient-specific PSA data, identifying the patient-specific parameters, calculating one or more predicted PSA values, and making a prediction.

In some arrangements, the personalized model utilizes the following equation:

$$P ( t ) = \{ \begin{matrix}{P}_{0} {e}^{{ρ}_{n} t} & t < {t}_{D} \\ {P}_{0} {e}^{{t}_{D} ( {ρ}_{n} - {ρ}_{s} )} [ {R}_{D} {e}^{{ρ}_{s} t} + ( 1 - {R}_{D} ) {e}^{{t}_{D} ( {ρ}_{s} + {ρ}_{d} )} {e}^{- {ρ}_{d} t} ] & t > {t}_{D}\end{matrix} .$$

In some arrangements, the personalized model utilizes the following equation:

$$P ( t ) = {P}_{0} {θ}_{1} ⌈ {R}_{d}^{{n}_{d}} {e}^{{ρ}_{s} t} + ( 1 - {R}_{d} ) ( {\sum }_{i = 1}^{{n}_{d}} {R}_{d}^{i - 1} {e}^{( {t}_{i} - {t}_{1} ) ( {ρ}_{s} + {ρ}_{d} )} ) {θ}_{2} {e}^{- {ρ}_{d} t} ⌉ , t > {t}_{{n}_{d}} .$$

According to yet another nonlimiting aspect, a software product includes a set of instructions on a non-transitory medium configured to cause a computer to implement the identifying step of the method described above, and to implement the calculating step of the method described above. Optionally, the software product may also include instructions configured to cause the computer to implement the predicting step.

According to still another nonlimiting aspect, a system for predicting relapse of a prostate cancer patient treated by radiation therapy includes a computer system comprising at least one processor configured to run the software product, and a user interface configured to provide to a user the predicted PSA values and/or whether and/or when a relapse is predicted to occur.

Technical aspects of methods and systems as described above preferably include the ability to recapitulate observed changes in PSA and yield short-term predictions capable of assisting in decision-making in post-radiation therapy monitoring of prostate cancer. In some arrangements, the methods, software, and/or systems may make it possible, earlier than in conventional methods, to more accurately predict whether and/or when a relapse of the prostate cancer patient will occur, determine an optimal frequency of future PSA tests for the patient, and/or choose a course of action in the post-treatment monitoring of a prostate cancer patient, such as selecting a frequency of future PSA tests and/or deciding whether to investigate a potential tumor recurrence.

These and other aspects, arrangements, features, and/or technical effects will become apparent upon detailed inspection of the figures and the following description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically represents mechanisms included in models of PCa response to EBRT according to certain nonlimiting aspects of the invention.

FIGS. 2A through 2F are graphs indicating that mechanistic models are capable of reproducing observed PSA dynamics during post-EBRT monitoring.

FIGS. 3A through 3H are graphs that indicate distributions of candidate model-based biomarkers of biochemical relapse obtained in a global fitting study.

FIGS. 4A through 4D are ROC curves for model-based biomarkers of biochemical relapse identified by analysis of the global fitting study results.

FIGS. 5A through 5I are graphs representing mechanistic models that provide personalized forecasts of PSA dynamics during post-EBRT monitoring.

FIGS. 6A through 6D are ROC curves indicating early estimates of biochemical relapse biomarkers obtained in fitting-forecasting studies.

FIG. 7 contains a graph indicating the distribution of days gained to biochemical relapse diagnosis (DGBRD) for model-based biomarkers of biochemical relapse.

### DETAILED DESCRIPTION OF THE INVENTION

The intended purpose of the following detailed description of the invention and the phraseology and terminology employed therein is to describe what is shown in the drawings, which relate to one or more nonlimiting embodiments of the invention, and to describe certain but not all aspects of the embodiment(s) to which the drawings relate. The following detailed description also describes certain investigations relating to the embodiment(s) depicted in the drawings, and identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

In some embodiments, the methods and systems disclosed herein provide a method and an algorithm that, in some configurations, may be used to predict relapse of prostate cancer patients treated by radiation therapy. The detection of prostate cancer recurrence after external beam radiotherapy (EBR) conventionally relies on the measurement of a sustained rise of serum prostate-specific antigen (PSA) that indicates the occurrence of biochemical relapse. However, this biochemical relapse may take years to occur, thereby delaying the delivery of a secondary treatment to patients with recurring tumors. To address this, some nonlimiting aspects of methods and systems disclosed herein provide a method to use patient-specific forecasts of PSA dynamics to predict biochemical relapse sooner than with conventional techniques. Forecasts obtained with this method are based on mechanistic models of prostate cancer response to external beam radiotherapy, which are fit to patient-specific PSA data collected during standard post-treatment monitoring. Experimental results from this method show good performance of the models in recapitulating the observed changes in PSA and yielding short-term predictions over approximately one year (cohort median RMSE of 0.10 to 0.47 ng/ml and 0.13 to 1.41 ng/ml, respectively). Additionally, some nonlimiting embodiments of the methods and systems disclosed herein identify three model-based biomarkers that enable an accurate identification of biochemical relapse (AUC>0.80) significantly earlier than standard practice (p<0.01).

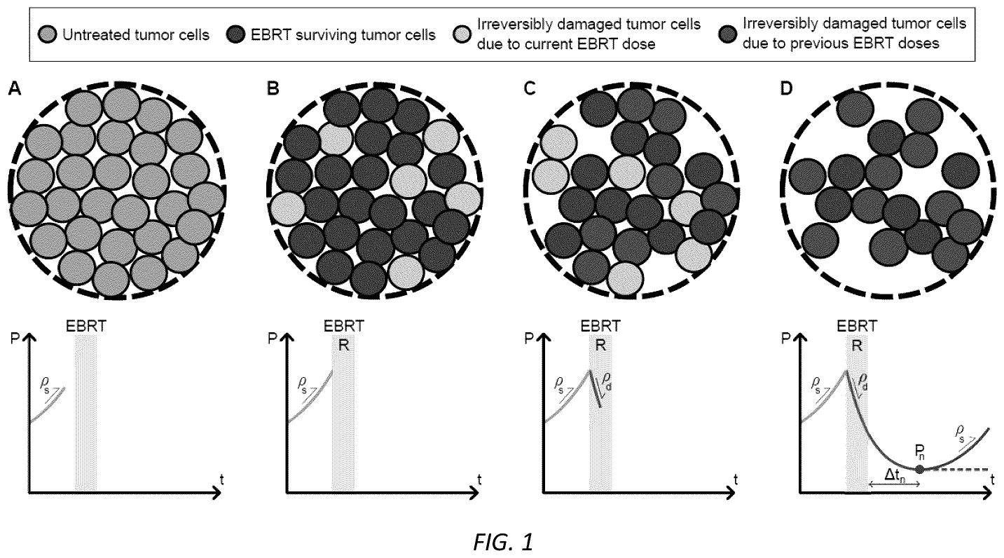

To address the limitations in the conventional methods discussed previously, the methods and systems disclosed herein may leverage mechanistic models of PCa response to EBRT (see FIG. 1) in order to forecast PSA dynamics on a patient-specific basis. This approach is used to predict the occurrence of biochemical relapse and thereby facilitate an early diagnosis and treatment of tumor recurrence after EBRT. The mechanistic modeling of tumor growth and therapeutic response is an established approach that aims at mathematically describing the biophysical mechanisms underlying these phenomena in order to increase understanding of cancer diseases and advance their clinical management on a personalized basis. In particular, these models can be fit to patient-specific data and then leveraged to render personalized computer forecasts of tumor prognosis and treatment outcomes capable to assist clinical decision-making.

According to some aspects of the methods and systems disclosed herein, a basic algorithm for predicting relapse of a prostate cancer patient is provided. In a first step (Step 1), nₚ measurements of serum prostate specific antigen (PSA) are collected, respectively, at nₚ dates for a newly-diagnosed prostate cancer patient before and after an external beam radiation therapy regimen prescribed as primary treatment for prostate cancer with curative intent. Preferably, at least 4 PSA measurements are obtained, of which at least one PSA measurement is collected before the radiotherapy begins, although fewer PSA measurements may be collected and/or used. This patient-specific set of PSA data can be extended as new PSA measurements are collected for the patient during the standard monitoring of prostate cancer before and after the radiation treatment.

In a second step (Step 2), the patient-specific parameters P₀, ρₙ, ρₛ, ρ_{d}, and R_{D} are identified that render an optimal fit of the patient-specific PSA data to a personalized model. In some arrangements, the personalized model uses the equation

$$P ( t ) = \{ \begin{matrix}{P}_{0} {e}^{{ρ}_{n} t} & t < {t}_{D} \\ {P}_{0} {e}^{{t}_{D} ( {ρ}_{n} - {ρ}_{s} )} [ {R}_{D} {e}^{{ρ}_{s} t} + ( 1 - {R}_{D} ) {e}^{{t}_{D} ( {ρ}_{s} + {ρ}_{d} )} {e}^{- {ρ}_{d} t} ] & t > {t}_{D}\end{matrix}$$

where:
P(t) represents the serum PSA value at time t;
Time t=0 corresponds to the first available PSA measurement for the patient;
Time t=t_{D} corresponds to the onset of radiotherapy;
Parameter P₀ represents the baseline PSA as measured prior to radiotherapy;
Parameter ρₙ represents the net proliferation rate of prostate cancer cells before radiotherapy;
Parameter ρₛ represents the net proliferation rate of the prostate cancer cells surviving the radiation treatment;
Parameter ρ_{d} represents the radiation-induced death rate of prostate cancer cells; and
Parameter R_{D} represents the fraction of prostate cancer cells that survive the radiation treatment.

However, the personalized model may use other equations, such as

$$P ( t ) = {P}_{0} {θ}_{1} [ {R}_{d}^{{n}_{d}} {e}^{{ρ}_{s} t} + ( 1 - {R}_{d} ) ( {\sum }_{i = 1}^{{n}_{d}} {R}_{d}^{i - 1} {e}^{( {t}_{i} - {t}_{1} ) ( {ρ}_{s} + {ρ}_{d} )} ) {θ}_{2} {e}^{- {ρ}_{d} t} ] , t > {t}_{{n}_{d}}$$

as discussed hereinafter.

In a third step (Step 3), the personalized model with the patient-specific parameters from Step 2 is used to calculate a prediction of the PSA values at several selected time horizons in the short term (e.g., up to 1 to 2 years after the last PSA measurement). These predictions can help physicians decide the best course of action in the post-treatment monitoring of prostate cancer patients (e.g., decide the frequency of future PSA tests, investigate a potential tumor recurrence if the predictions of PSA exhibit a rising trend). Preferably, a panel of four model-based biomarkers of biochemical relapse (i.e., a consistent rising trend in PSA dynamics after radiotherapy caused by an underlying tumor recurrence), which aim at early detection of this phenomenon, may be calculated. These model-based biomarkers are the proliferation rate of the surviving prostate cancer cells ρₛ, the ratio of the proliferation rate of the surviving prostate cancer cells and the radiation-induced death rate of prostate cancer cells β=ρₛ/ρ_{d}, the PSA nadir Pₙ (i.e., the minimum PSA value predicted by the model after the conclusion of radiotherapy), and the time to PSA nadir after the termination of radiotherapy Δtₙ. The patient-specific values of these biomarkers are calculated to corresponding population-based thresholds to classify the patient as relapsing or non-relapsing.

Thereafter, it can be predicted if and/or when a relapse of the prostate cancer patient will occur as a function of the one or more predicted PSA values. For example, in some arrangements, the predicted critical PSA value may be a nadir of the plurality of PSA values obtained as a function of the personalized model and the patient-specific parameters during the selected time horizon.

In a fourth step (Step 4), when a new PSA measurement is collected for the patient, the model parameters and ensuing predictions may optionally be updated by repeating Steps 1-3.

In view of this general basic algorithm, in a first embodiment, a first method (method 1) is provided to predict post-radiotherapy short-term PSA dynamics after the last measurement of PSA on a patient-specific basis and determine the values of the model-based biomarkers to classify the patient as relapsing or non-relapsing. This method corresponds to the application of steps 1-4 of the basic algorithm described above.

In a second embodiment (method 2), method 1 described above is cast in a Bayesian framework, where the prior distributions of the parameters are determined from the fitting of the model to all available PSA data from each patient in a population, where the prior distributions of the measurement errors are estimated from previous studies in the literature, and where Bayesian inference is used to determine the posterior distributions of model parameters, model predictions of PSA, and the probabilistic risk of biochemical relapse according to the model-based biomarkers.

In a third embodiment (method 3), in either of the methods 1 or 2 described above, an extra step after step 3 is performed, in which the model predictions of PSA are leveraged to determine the optimal frequency of future PSA tests for each patient, such that the number of tests is minimal (using Methods 1 or 2), the accuracy of the model predictions is maximal (using Methods 1 and 2), and the probabilistic risk of late detection of biochemical relapse is minimal (using Method 2).

In a fourth embodiment (method 4), in either of the methods 1 and 2 described above, only the PSA values collected before radiotherapy inform the personalized model and the ensuing model predictions of radiotherapy outcome considering admissible values of the parameters governing radiation response (i.e., ρₛ, ρ_{d}, and R_{D}) are leveraged to optimize the radiotherapeutic plan for an individual patient (i.e., total dose, treatment onset date), such that it minimizes the PSA after radiotherapy to a flat trend with a small constant value and using a minimal radiation dose (methods 1 and 2). In Method 2, this objective may be cast in a probabilistic formulation that ultimately aims at minimizing the probabilistic risk of biochemical relapse after treatment using a minimal dose.

In a fifth embodiment (method 5), in the method 4 described above, the personalized model is replaced by the parent formulation described hereinafter, which further accounts for the dates of radiation treatment and, hence, makes it possible for method 4 to extend by further enabling the optimization of the radiation dose per fraction and the frequency of the radiation dose.

In a sixth embodiment (method 6), in any of the methods 1 to 5 described above, the personalized model is extended by adding an additional positive exponential rate representing the increase in BPH caused by coexisting benign prostatic hyperplasia (BPH), which is estimated from population studies (i.e., \hat{P}(t)=P(t)+e^{ρ}^{_{BPH}}^{t}).

Various aspects of the method are suitable for being executed by a software product on a computer system. For example, a software product according to some aspects of the invention may include a set of instructions on a non-transitory medium configured to cause a computer system to implement the step of identifying any one or more of the patient-specific parameters. The software product may also be configured to cause a computer system to implement the step of calculating the set of predicted PSA value(s) at a selected time horizon(s) after the PSA measurements. The set of instructions may also be configured to cause the computer system to implement the step of predicting if and/or when a relapse of the prostate cancer patient will occur. The software product is preferably stored on a non-transient medium, such as a floppy disc, compact disc, thumb drive, hard drive, etc.

The method and software of the present invention may be implemented as part of a computerized system for predicting relapse of a prostate cancer patient treated by radiation therapy. Such a computer system may include at least one processor configured to execute the software product described above. The processor(s) may include, for example, processors on a personal computer, a handheld computing device, cloud-based computing device(s), other types of digital and/or quantum processors, and/or combinations thereof. The computer system may include multiple processors interconnected, for example by a wired and/or wireless network, such as a local area network, wide area network, and/or internet. The system preferably also includes a user interface, such as a display screen, printer, etc., that is configured to provide to a user the predicted PSA values and/or whether and/or when a relapse is predicted to occur.

In a prior study, a mechanistic modeling framework was presented to describe how the response of PCa to EBRT drives PSA dynamics after treatment and identified promising model-based biomarkers to detect biochemical relapse. The modeling framework relies on five key assumptions that are illustrated in FIG. 1. It is assumed that PCa cells 10 proliferate following an exponential law and that PSA is proportional to the number of tumor cells. It is also assumed that each radiation dose irreversibly damages a fraction of the tumor cells that ultimately undergoes programmed cell death, whereas the complementary fraction survives the EBRT and continues proliferating. Additionally, it is assumed that EBRT is delivered either periodically or as an equivalent single dose. This last assumption leads to two alternative models that are termed as the periodic dose model and the single dose model, respectively. (See STAR Methods for further details on mathematical modeling).

FIG. 1 relates to the mechanistic modeling of PSA dynamics after EBRT for the present methods and systems and illustrates the mechanisms included in the models of PCa response to EBRT, which assume that serum PSA is proportional to the number of cells in the patient's tumor. The upper row shows the PCa cells in a generic tumor region at four instants in time (A, B, C, and D) before, during, and after EBRT. The bottom row shows the corresponding PSA evolution up to each depicted time instant. At time (A), before treatment, it is assumed that tumor cells grow exponentially at a rate ρₛ, which also describes the characteristic increase of PSA in untreated PCa (rose solid line). At time (B), after the first EBRT dose, a fraction of tumor cells R survives to radiation, while the complementary fraction, 1−R, is irreversibly damaged and will ultimately die. This process will repeat with each consecutive EBRT dose. At time (C), during EBRT, while the surviving cells may continue to proliferate at a rate ρₛ, the radiation-damaged cells undergo programmed cell death at a rate ρ_{d}. This becomes the dominant mechanism during EBRT, and produces a decreasing trend in PSA (gray solid line). At time (D), after the conclusion of EBRT, the remainder of the radiation-damaged cells die and PSA continues to progressively drop. If the EBRT treatment does not fully eliminate the tumor, the surviving cells continue proliferating at a rate ρₛ and ultimately produce a biochemical relapse (red solid line). If the treatment eradicates all tumor cells, then PSA reaches a plateau (gray dashed line). The mechanistic models of the methods and systems disclosed herein make it possible to calculate a quantitative estimation of the PSA nadir (Pₙ) and the time to PSA nadir since EBRT termination (Δtn). The STAR Methods described hereinafter provide further details on the mathematical modeling.

In studies leading to the present invention, models and model-based biomarkers of biochemical relapse were validated from prior research reported in Lorenzo et al. “Mechanistic modelling of prostate specific antigen dynamics shows potential for personalized prediction of radiation therapy outcome,” J. R. Soc. Interface 16: 20190195 (available at http://dx.doi.org/10.1098/rsif.2019.0195) (referred herein as “Lorenzo et al. (2019)”), the contents of which are incorporated herein by reference, in a new cohort, whose main characteristics are summarized in Table 1. (See STAR Methods for further details.) Table 1 is a summary of patient cohort characteristics used in studies related to the present invention. To this end, a global fitting study is performed in which the models with all PSA data available for each patient are parameterized. Then, the predictive performance of the models is assessed in a series of fitting-forecasting scenarios. Each scenario leverages an increasing number of the PSA values collected for each patient for model fitting, which is followed by a corresponding personalized model forecast of PSA dynamics that is compared against the remainder of the patient's PSA data. This approach would simulate the utilization of the models during actual patient monitoring: each newly collected PSA value enables the models for a given patient to be updated and, hence, obtain an updated prediction of PSA dynamics on an individual basis. Additionally, the ability of the model-based biomarkers is analyzed as determined in these fitting-forecasting scenarios to early identify biochemical relapse, and it is further assessed whether they outperform the standard clinical criteria that were used in this cohort.

TABLE 1

| | All patients (n = 166) | | | Non-relapsing patients (n = 156) | | | Relapsing patients (n = 10) | | |
|---|---|---|---|---|---|---|---|---|---|
| Characteristic | Median | IQR | Range | Median | IQR | Range | Median | IQR | Range |
| Clinical | | | | | | | | | |
| Age at diagnosis (y) | 73 | (68, 76) | (49, 83) | 73 | (68, 76) | (49, 83) | 73 | (68, 76) | (65, 79) |
| Baseline PSA, P_{d} (ng/mL) | 6.3 | (4.6, 8.6) | (0.6, 81.0) | 6.2 | (4.5, 8.4) | (0.6, 22.0) | 9.6 | (5.9, 13.1) | (4.9, 81.0) |
| Gleason score | 6 | (6, 7) | (4, 8) | 6 | (6, 7) | (5, 8) | 7 | (6, 7) | (4, 7) |
| EBRT | | | | | | | | | |
| Age at EBRT (y) | 74 | (68, 77) | (49, 84) | 74 | (68, 77) | (49, 84) | 75 | (73, 76) | (66, 80) |
| Total dose (Gy) | 74.2 | (71.4, 74.2) | (65.8, 77.4) | 74.2 | (71.4, 74.2) | (71.4, 77.4) | 74.2 | (71.4, 74.2) | (65.8, 74.2) |
| Dose per fraction (Gy) | 2.65 | (2.55, 2.65) | (2.35, 2.76) | 2.65 | (2.55, 2.65) | (2.55, 2.76) | 2.65 | (2.55, 2.65) | (2.35, 2.65) |
| Duration (mo) | 1.3 | (1.3, 1.4) | (1.2, 2.1) | 1.3 | (1.3, 1.4) | (1.2, 2.1) | 1.3 | (1.3, 1.4) | (1.2, 1.7) |
| Patient monitoring | | | | | | | | | |
| No. PSA values, nₚ | 11 | (8, 13) | (6, 25) | 11 | (8, 13) | (6, 25) | 14 | (10, 19) | (7, 22) |
| Follow-up since EBRT (y) | 5.7 | (4.5, 7.6) | (3.0, 14.0) | 5.7 | (4.5, 7.5) | (3.0, 14.0) | 5.7 | (4.2, 8.1) | (3.3, 11.2) |
| PSA test frequency (mo) | 6.3 | (4.0, 10.3) | (0.0, 59.4) | 6.4 | (4.1, 11.4) | (0.0, 59.4) | 4.9 | (3.2, 7.3) | (1.0, 20.5) |

STAR Method: In the STAR method according to aspects of the methods and systems disclosed herein a general mathematical model is used as described hereinafter.

General mathematical model: P(t) is the serum PSA at time t. The time interval of interest is (t₀,t_{f}), where to is the time at which the pre-EBTR PSA measurement in the database was taken, and t_{f} is the latest time at which the PSA is forecasted. For simplicity, time is rescaled such that t₀=0 for all patients. The patients in the database received n_{d} radiation doses at times {tₖ}_{k=1, . . . nd}, where t₀<t₁< . . . t_{nd}<t_{f}. The tₖ's may vary from patient to patient.

It is assumed that the serum PSA is proportional to the number of tumor cells N(t), that is

$$P ( t ) = κ N ( t ) , \qquad ( 1 )$$

Where λ is a constant. Prior to EBRT treatment, it is assumed that N grows exponentially in time from N(t₀)=N₀, at a characteristic rate ρₙ. Thus,

$$P ( t ) = κ {N}_{0} {e}^{{ρ}_{n} t} in t ∈ ( {t}_{0} , {t}_{1} ) . \\ From Eq . ( 2 ) , {P}_{0} = P ( 0 ) = κ {N}_{0} . \qquad ( 2 )$$

For each time interval, Iₖ=(tₖ,tₖ₊₁), k=1, . . . , n_{d}−1, Sₖ(t) represents the fraction of tumor cells surviving to the k-th radiation dose, and \tilde{D}ₖ(t) is the fraction of tumor cells irreversibly damaged after the k-th radiation dose. For compactness of the notation, S₀(t) and \tilde{D}₀(t) in the interval (t₀,t₁) are S₀(t)=N(t) and \tilde{D}₀(t)=0. This assumes that there are no damaged cells before treatment.

The values of Sₖ and \tilde{D}ₖ at time tₖ are obtained from and \tilde{D}ₖ₋₁ and Sₖ₋₁ as

$${\tilde{D}}_{k} ( {t}_{k} ) = ( 1 - {R}_{d} ) {S}_{k - 1} ( {t}_{k} ) , \qquad ( 3 a )$$

$${S}_{k} ( {t}_{k} ) = {R}_{d} {S}_{k - 1} ( {t}_{k} ) . \qquad ( 3 b )$$

Eq. (3a) assumes that the radiation dose at time/immediately produces irreversible damage to a fraction of cells (1−R_{d}), where 0<R_{d}<1. The remaining fraction of cells, R_{d}, continues in the compartment of surviving cells. The parameter R_{d} is patient specific, but constant for all doses. No specific formulation is assumed for R_{d}, but rather R_{d} is computed from the PSA data. Eqs. (3a)-(3b) provide initial conditions for the ordinary differential equations (ODEs) that govern the dynamics of \tilde{D}ₖ(t) and Sₖ(t) in the time interval Iₖ. These ODEs are based on the assumptions that irreversibly damaged cells undergo programmed cell death at a rate ρ_{d}, and surviving cells continue their proliferation at a characteristic rate ρₛ,

$$\frac{d {\tilde{D}}_{k}}{dt} = - {ρ}_{d} {\tilde{D}}_{k} in {I}_{k} , \qquad ( 4 a )$$

$$\frac{{dS}_{k}}{dt} = {ρ}_{s} {S}_{k} in {I}_{k} . \qquad ( 4 b )$$

The cumulative number of irreversibly damaged tumor cells in the time interval Iₖ is defined as

$${D}_{k} ( t ) = {D}_{k - 1} ( {t}_{k} ) + {\tilde{D}}_{k} ( t ) , \qquad ( 5 )$$

with D₀(t)=0. Hence, the total population of tumor cells in the interval Iₖ is

$${N}_{k} ( t ) = {S}_{k} ( t ) + {D}_{k} ( t ) , \qquad ( 6 )$$

and the PSA is given by

$${P}_{k} ( t ) = κ {N}_{k} ( t ) = κ ( {S}_{k} ( t ) + {D}_{k} ( t ) ) , \qquad ( 7 )$$

Eqs. (3a) (4b) can be solved recursively on all time intervals from I₁ to I_{nd-1} through direct integration, which leads to

$${\tilde{D}}_{k} ( t ) = ( 1 - {R}_{d} ) {S}_{k - 1} ( {t}_{k} ) {e}^{- {ρ}_{d} ( t - {t}_{k} )} in {I}_{k} , \qquad ( 8 a )$$

$${S}_{k} ( t ) = {R}_{d} {S}_{k - 1} ( {t}_{k} ) {e}^{{ρ}_{s} ( t - {t}_{k} )} in {I}_{k} . \qquad ( 8 b )$$

From Eqs. (8a) and (5), it can be obtained that

$${D}_{k} ( t ) = {D}_{k - 1} ( {t}_{k} ) + ( 1 - {R}_{d} ) {S}_{k - 1} ( {t}_{k} ) {e}^{- {ρ}_{d} ( t - {t}_{k} )} in {I}_{k} . \qquad ( 9 )$$

By applying Eqs. (8b) and (5) recursively, it can be derived that

$${S}_{k} ( t ) = {R}_{d}^{k} {N}_{0} {θ}_{1} {e}^{{ρ}_{s} t} in {I}_{k} , \qquad ( 10 a )$$

$${D}_{k} ( t ) = ( 1 - {R}_{d} ) [ {\sum }_{i = 1}^{k} {R}_{d}^{l - 1} {e}^{( {i}_{i} - {i}_{1} ) ( {ρ}_{s} + {ρ}_{d} )} ] {N}_{0} {θ}_{1} {θ}_{2} {e}^{- {ρ}_{d} t} in {I}_{k} , \qquad ( 10 b )$$

where θ₁=e^{t1(ρn-ρs)} and θ₂=e^{t1(ρs+ρd)}. Then, the expression for the serum PSA is

$${P}_{k} ( t ) = {P}_{0} {θ}_{1} [ {R}_{d}^{k} {e}^{{ρ}_{s} t} + ( 1 - {R}_{d} ) ( {\sum }_{i = 1}^{k} {R}_{d}^{i - 1} {e}^{( {i}_{i} - {i}_{1} ) ( {ρ}_{s} + {ρ}_{d} )} ) {θ}_{2} {e}^{- {ρ}_{d} t} ] in {I}_{k} . \qquad ( 11 )$$

Periodic dose model: In the periodic dose model, it is assumed that the radiation doses are evenly spaced in time with a constant intertreatment interval τᵣ, i.e., tₖ=t₁+(k−1)τᵣ for k=1, . . . , n_{d}. This allows Eq. (10b) to be simplified into

$${D}_{k} ( t ) = ( 1 - {R}_{d} ) \frac{1 - {R}_{d}^{k} {e}^{k τ} {\text{?}}^{( {ρ}_{s} + {ρ}_{d} )}}{1 - {R}_{d} {e}^{τ} {\text{?}}^{( {ρ}_{s} + {ρ}_{d} )}} {N}_{0} {θ}_{1} {θ}_{2} {e}^{- {ρ}_{d} t} in {I}_{k} . \qquad ( 12 )$$

$$\text{?} \text{indicates text missing or illegible when filed}$$

Then, the PSA is given by

$${P}_{k} ( t ) = {P}_{0} {θ}_{1} [ {R}_{d}^{k} {e}^{ρ , t} + ( 1 - {R}_{d} ) \frac{1 - {R}_{d}^{k} {e}^{k {τ}_{r} ( {ρ}_{s} + {ρ}_{d} )}}{1 - {R}_{d} {e}^{{τ}_{r} ( {ρ}_{s} + {ρ}_{d} )}} {θ}_{2} {e}^{- ρ} \text{?} ] in {I}_{k} . \qquad ( 13 )$$

$$\text{?} \text{indicates text missing or illegible when filed}$$

Single dose model: In the single dose model, it is assumed that the entire radiation treatment is given in one single dose at time t_{D}. Then,

$$S ( t ) = {R}_{D} {N}_{0} {θ}_{1 D} {e}^{ρ} \text{?} , t > {t}_{D} , \qquad ( 14 a )$$

$$D ( t ) = ( 1 - {R}_{D} ) {N}_{0} {θ}_{1 D} {θ}_{2 D} {e}^{- {ρ}_{d} t} , t > {t}_{D} , \qquad ( 14 b )$$

$$\text{?} \text{indicates text missing or illegible when filed}$$

Where R_{d} is the fraction of surviving tumor cells after the entire treatment dose, θ_{1D}=e^{tD(ρn-ρs)} and θ_{2D}=e^{tD(ρs+ρd)}. Then, the PSA is given by

$$P ( t ) = {P}_{0} {θ}_{1 D} [ {R}_{D} {e}^{ρ} \text{?} + ( 1 - {R}_{D} ) {θ}_{2 D} {e}^{- ρ} \text{?} ] . \qquad ( 15 )$$

$$\text{?} \text{indicates text missing or illegible when filed}$$

Dimensional analysis and predicted PSA nadir: The PSA dynamics after treatment completion can be obtained from Eq. (13) as

$$P ( t ) = {P}_{0} {θ}_{1} [ {R}_{d}^{{n}_{d}} {e}^{ρ} \text{?} + ( 1 - {R}_{d} ) ( {{\sum }^{{n}_{d}}}_{i = 1} {R}_{d}^{i - 1} {e}^{( {t}_{i} - {t}_{1} ) ( ρ} {\text{?}}^{+ {ρ}_{d} )} ) {θ}_{2} {e}^{- ρ} \text{?} ] , t > {t}_{{n}_{d}} . \qquad ( 16 )$$

$$\text{?} \text{indicates text missing or illegible when filed}$$

PSA and time are nondimensionalized using the scales P₀θ₁(1−R_{d}) (Σᵢ₌₁ⁿ^{_{d}}R_{d}ⁱ⁻¹e^{(t}^{ᵢ}^{-t}^{₁}^{)(ρ}^{ₛ}^{+ρ}^{_{d}}⁾) and 1/ρ_{d}, respectively. Then, using hats to denote dimensionless quantities,

$$\hat{P} ( \hat{t} ) = \frac{{R}_{d}^{{n}_{d}}}{( 1 - {R}_{d} ) {\sum }_{i = 1}^{{n}_{d}} {R}_{d}^{i - 1} e {\text{?}}^{+ 1 )}} e \text{?} + {θ}_{2} {e}^{-} \text{?} . \qquad ( 17 )$$

$$\text{?} \text{indicates text missing or illegible when filed}$$

Defining the nondimensional groups

$$β = \frac{{ρ}_{s}}{{ρ}_{d}} , \qquad ( 18 a )$$

$$α = \frac{{R}_{d}^{{n}_{d}}}{( 1 - {R}_{d} ) {\sum }_{i = 1}^{{n}_{d}} {R}_{d}^{i - 1} e {\text{?}}^{+ 1 )}} , \qquad ( 18 b )$$

$$\text{?} \text{indicates text missing or illegible when filed}$$

the dimensionless PSA can be expressed as

$$\hat{P} ( \hat{t} ) = α {e}^{β} \text{?} + {θ}_{2} {e}^{-} \text{?} . \qquad ( 19 )$$

$$\text{?} \text{indicates text missing or illegible when filed}$$

The parameter α represents the efficacy of the radiation plan and β defines the dynamics of the tumor cell population after radiation. While β is independent from the treatment plan, α can be specialized to the two treatment plans studied in this paper. For the periodic dose treatment

$$α = \frac{{R}_{d}^{{n}_{d}}}{1 - {R}_{d}} \frac{1 - {R}_{d} {e}^{τ} {\text{?}}^{( {ρ}_{s} + {ρ}_{d} )}}{1 - R \text{?} e {\text{?}}^{+ {ρ}_{d} )}} , \qquad ( 20 )$$

$$\text{?} \text{indicates text missing or illegible when filed}$$

while for the single dose treatment

$$α = \frac{{R}_{D}}{1 - {R}_{D}} . \qquad ( 21 )$$

The nondimenzionalized PSA velocity is defined as

$$\hat{{v}_{P}} ( \hat{t} ) = \frac{d \hat{P} ( \text{?} )}{d \hat{t}} = αβ {e}^{β} \text{?} - {θ}_{2} {e}^{-} \text{?} . \qquad ( 22 )$$

$$\text{?} \text{indicates text missing or illegible when filed}$$

The PSA nadir is achieved at a dimensionless time \hat{t}ₙ defined by \hat{ν}_{P}(\hat{t}ₙ)=0. Using Eq. (22), it is found that

$${\hat{t}}_{n} = \frac{1}{1 + β} ln ( \frac{{θ}_{2}}{αβ} ) . \qquad ( 23 )$$

The corresponding dimensional time is

$${t}_{n} = {t}_{1} + \frac{1}{{ρ}_{d} ( 1 + β )} ln ( \frac{1}{αβ} ) . \qquad ( 24 )$$

and the time to PSA nadir since the completion of the treatment is given by Δtₙ=tₙ−tₙ_{_{d}}.

Further modeling assumptions: Some patients may experience delays in their radiation plans due to treatment side effects, local holidays, or machine routine maintenance. Additionally, the patient dataset used in this study only includes the times of EBRT initiation and conclusion, the radiation dose, and the number of doses. This information is not compatible with an accurate use of the general model, which would require the exact dates of each EBRT fraction for each patient. To overcome this limitation, the assumptions on radiation delivery that lead to the periodic and single dose models presented above are introduced. Thus, the analyses with these two models, which can be leveraged as surrogates of the general model as shown in Lorenzo et al. (2019) are performed.

It is further assumed that EBRT does not change the proliferation rate of the surviving tumor cells, such that ρₙ=ρₛ and θ₁=θ₁D=1. This is a common assumption in the literature that facilitates the parameterization of the models using the cohort of this study, which only features one pre-EBRT PSA value to inform ρₙ. Additionally, t_{D}=t₁ for the single dose model as in Lorenzo et al. (2019).

TABLE 2

| | | Initial | Lower | Upper |
|---|---|---|---|---|
| Parameter | Units | value | bound | bound |
| P0 | (ng/mL) | Pd | 0 | 100 |
| Rd | — | 0.9 | 0.5 | 1 |
| RD | — | 0.9nd | 0.5nd | 1 |
| ρs | (1/mo) | 0.02 | 0.001 | 2 |
| ρd | (1/mo) | 0.5 | 0.001 | 2 |

Model fitting and forecasting: the PSA data from each patient is fitted to both the periodic and single dose models. Model fitting is performed by leveraging a nonlinear least-squares method based on a trust-region reflective algorithm. Table 2 provides the initial guess as well as the lower and upper bounds of the model parameters for both the periodic and the single dose models. P_{d} is the baseline PSA value for each patient (see Table 1). The model fitting method aims at minimizing an objective functional J, given by

$$J = {{\sum }^{\text{?}}}_{i = 1} {( \hat{P} ( {t}_{i} ) - P ( {t}_{i} ) )}^{2} + {{w}_{r} ( {ρ}_{s} - {ρ}_{s , min} )}^{2} . \qquad ( 25 )$$

$$\text{?} \text{indicates text missing or illegible when filed}$$

The first term in the right-hand side of Eq. (25) represents the mismatch between PSA data (\hat{P}) and the model estimation of PSA (P) at each of the PSA test times tᵢ(i=1, . . . , n_{P,fit}). For each model and patient, a total of n_{P,fit} model fits in the fitting-forecasting study (n_{P,fit}=5, . . . , n_{P-1}) are run, whereas only a single model fit with n_{P,fit}=n_{P} is performed in the global fitting study. The second term in the right-hand side of Eq. (25) regularizes the proliferation rate of the surviving tumor cells (ρₛ) to low values and was introduced to limit overfitting, especially when only a small number of PSA values were available to calculate the model parameters. The regularization weight wᵣ is empirically set at 2500 and ρ_{s,min} is the minimum admissible value for this parameter in Table 2.

Further the model fitting problem is cast within a multi-start strategy to facilitate convergence for all patient datasets and avoid the selection of local minima in the minimization problem outlined above. In brief, this strategy consists of solving the model fitting problem multiple times, each time using a different initial guess and resulting in a parameter set if convergence is achieved. Then, the algorithm selects the resulting parameter set that renders the lowest value of the objective functional as the global minimum. A collection of 20 initial guesses is used that are randomly sampled within the parameter space and always includes the one provided in Table 2.

Code implementation: The calculations based on the numerical methods described in this section are performed using MAT-LAB. In particular, model fitting is implemented by leveraging the Global Optimization Toolbox.

Key resources, including the de-identified patient data, the MATLAB scripts for model fitting and forecasting, and the MATLAB scripts for statistical analysis used for this invention may be found at https://doi.org/10.5281/zenodo.6277674, the contents of which are incorporated by reference herein it its entirety.

Quantitative assessment of model fits and forecasts: In the global fitting study, the quality of fit is assessed by means of the root mean squared error (RMSE) and the coefficient of determination (R²). Given that some n_{P,fit} scenarios involved a reduced number of PSA values to assess the model forecasts, only the RMSE is used to analyze the quality of fit and validate the model predictions of PSA dynamics in the fitting-forecasting study. Additionally, the 95% nonlinear regression prediction confidence intervals are calculated for the model fits and forecasts.

Receiver operating characteristic curves: the receiver operating characteristic (ROC) curves are calculated for the model-based biomarkers obtained from global fitting and the fitting-forecasting study with either PSA model. Global fitting produces a unique value for each model-based biomarker per patient. The resulting set of values obtained across the whole cohort are used as the thresholds to construct the corresponding ROC curves. Since all PSA values for each patient are used in global fitting, these ROC curves assess the ability of the model-based biomarkers to retrospectively classify patients as relapsing or non-relapsing. The fitting-forecasting study produces a set of values for each model-based biomarker per patient, which result from the sequential model fits to an increasing number of PSA values ranging from n_{P,fit}=5 to n_{P,fit}=n_{P-1}. Hence, each set contains the temporal evolution of each model-based biomarker during post-EBRT monitoring for each patient. To construct the ROC curve of each model-based biomarker in the fitting-forecasting study, first all the biomarker values across all patients are pooled to define the thresholds. Then, for each patient, it is assessed whether each threshold can identify any of the biomarker values obtained across the n_{P,fit} scenarios as predictive for biochemical relapse. Thus, in this scenario, the ROC curves provide an assessment of the ability of the model-based biomarkers to early classify the patients as relapsing or non-relapsing during the course of post-EBRT PSA monitoring.

For each ROC curve, the area under the curve (AUC) is calculated using the trapezoidal rule and the optimal performance point according to Youdens index. Additionally, the 95% boot-strap confidence intervals of the ROC curves and their corresponding AUC and optimal performance point are calculated. 2000 bootstrap samples are used for these calculations. The 95% bootstrap confidence interval for each ROC curve is obtained as the envelope of the 95% bootstrap confidence interval regions obtained for each threshold value used in the construction of the ROC curve along the sensitivity and specificity axes.

Statistical analysis: Wilcoxon rank-sum and signed-rank tests are used for the statistical analyses performed in the investigations. As reported herein, it is specified when each type of test is used and whether it is two-tailed or one tailed for each statistical analysis of the global fitting and fitting-forecasting study results. The level of significance for all statistical tests is set at 5%.

Code implementation: The calculations based on the statistical methods described in this section are performed using MAT-LAB. In particular, the Statistics and Machine Learning Toolbox is used to calculate the 95% nonlinear regression prediction confidence intervals for the model fits and forecasts, construct the 95% bootstrap confidence intervals for the ROC curve analysis, and perform the aforementioned statistical tests.

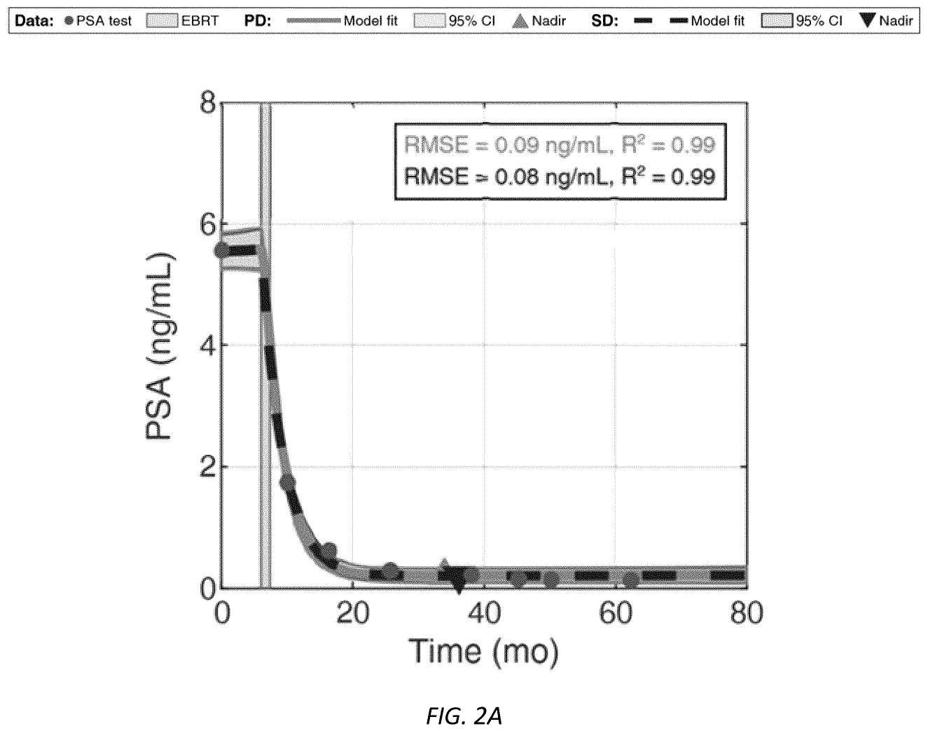

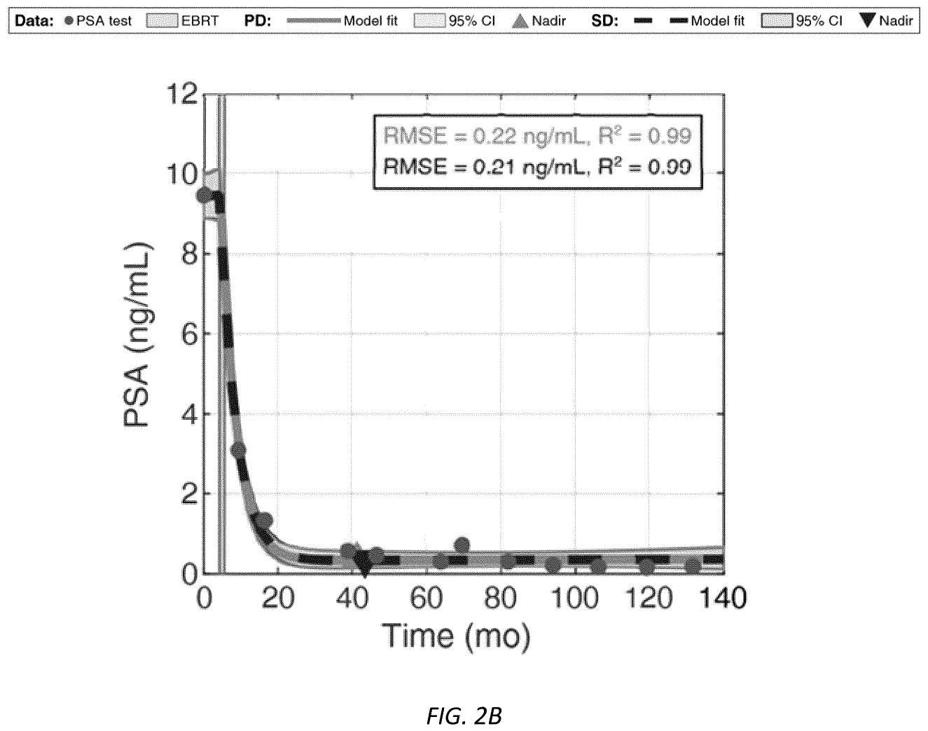

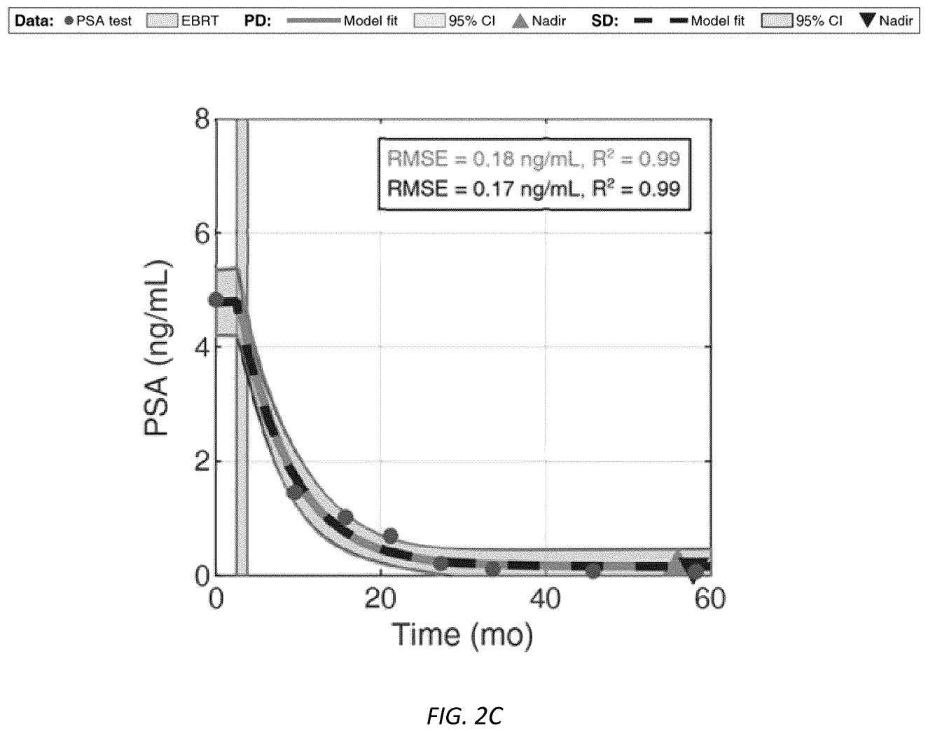

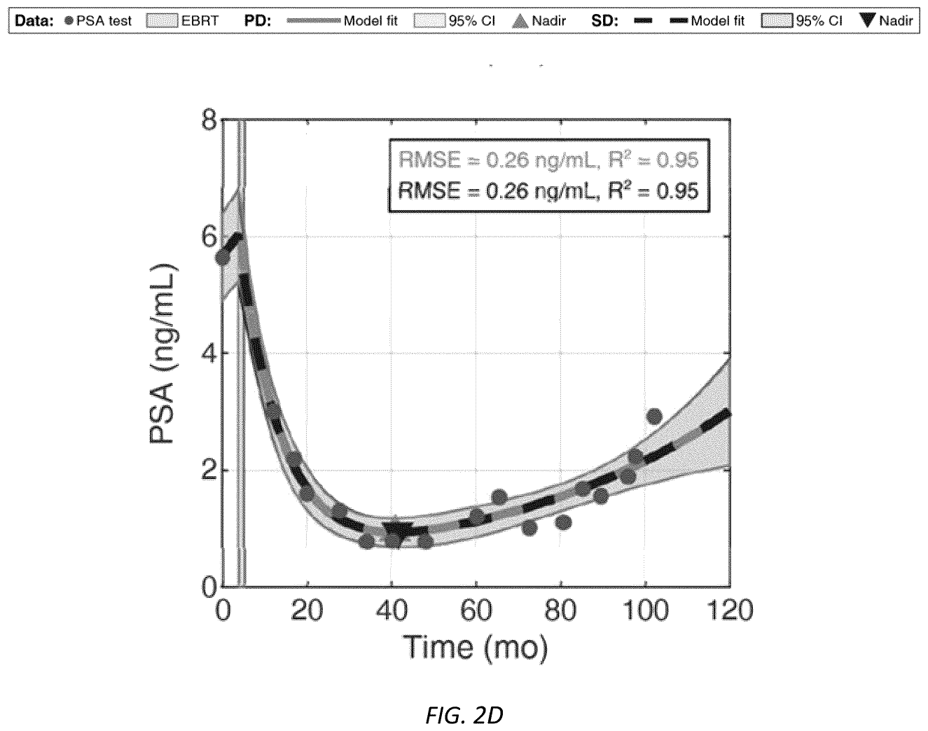

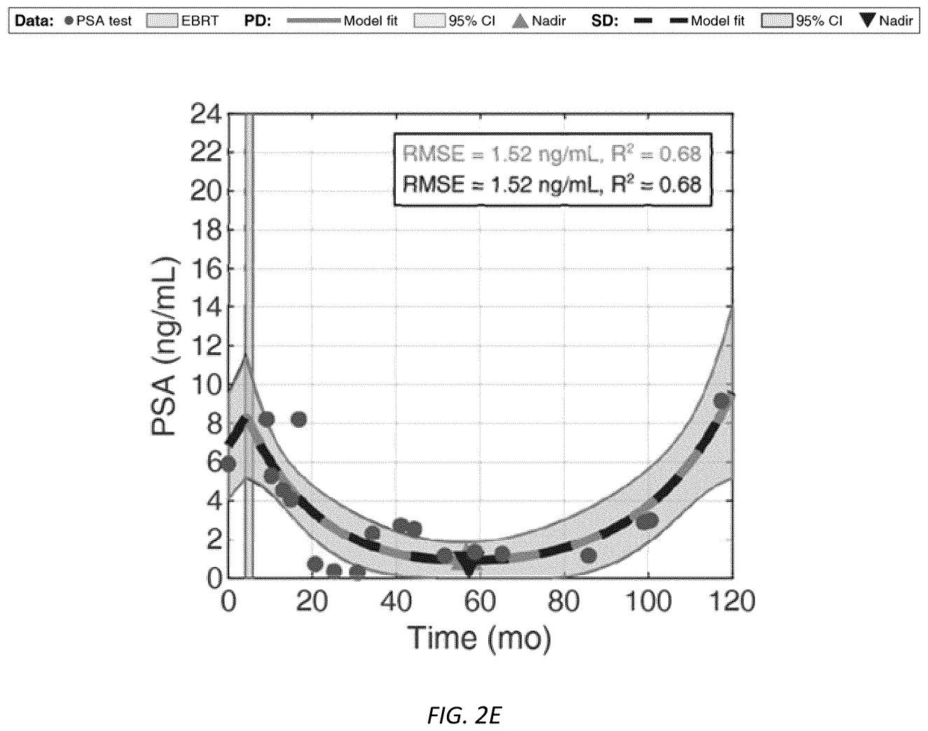

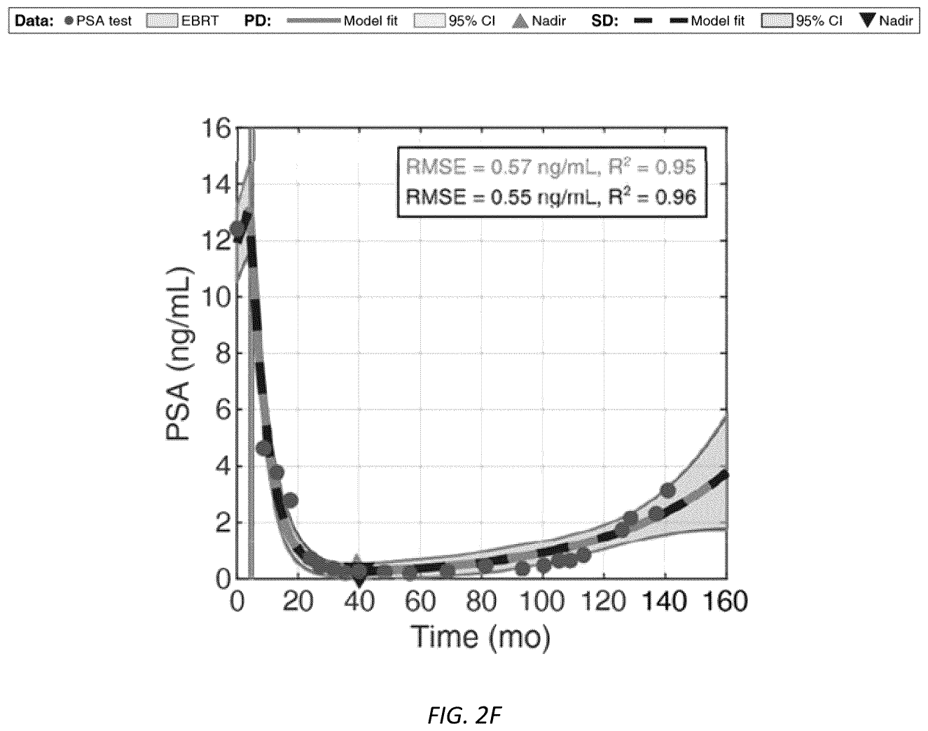

Mechanistic models recapitulate patient-specific PSA dynamics after EBRT. To begin, a global fitting analysis is performed to assess the ability of the mechanistic models in reproducing the longitudinal PSA series collected for each patient in the cohort. To this end, the periodic dose model and the single dose model are fitted to all the PSA values available for each patient (see STAR Methods for methodological details). FIG. 2 illustrates representative global fitting results for three non-relapsing and three relapsing patients. Using the periodic dose model, the median and interquartile range of the root mean squared error (RMSE) and the coefficient of determination (R²) of the model fits are 0.17 (0.08, 0.28) ng/ml and 0.99 (0.97, >0.99) over the whole cohort (n=166), 0.15 (0.07, 0.24) ng/ml and 0.99 (0.98, >0.99) in the non-relapsing subgroup (n=156), and 0.53 (0.40, 0.59) ng/ml and 0.95 (0.91, 0.98) in the relapsing subgroup (n=10), respectively. For the single-dose model, the RMSE and R² of the model fits are distributed with median and interquartile range of 0.16 (0.07, 0.26) ng/mL and 0.99 (0.98, >0.99) over the whole cohort (n=166), 0.15 (0.07, 0.23) ng/ml and 0.99 (0.98, >0.99) in the non-relapsing subgroup (n=156), and 0.53 (0.39, 0.59) ng/ml and 0.95 (0.92, 0.98) in the relapsing subgroup (n=10), respectively. Thus, the results from global fitting analysis demonstrate that the periodic dose model and the single dose model successfully recapitulate the observed patient-specific PSA dynamics. in FIGS. 2A through 2F also show a good agreement between the fits provided by either model.

In FIGS. 2A through 2F, FIGS. 2A-2C show global fitting results for three non-relapsing patients, while FIGS. 2D-2F show similar results for three patients exhibiting a biochemical relapse. For each patient, the PSA measurements are plotted as red bullet points and EBRT is represented as a rectangular area shaded in light gray. The fits obtained with the periodic dose model (PD) are represented with a solid green line, while the fits produced by the single dose model (SD) are depicted as a dashed blue line. The same color scheme is also used for the 95% confidence intervals, which are depicted as the shaded areas around the model fits, and the font of the quality of fit metrics for either model. Additionally, each plot also includes the prediction of the patient's PSA nadir using the periodic dose model (green upward triangle) and the single dose model (blue downward triangle). This figure illustrates the remarkable performance of the mechanistic models to fit patient-specific longitudinal PSA datasets collected during standard monitoring following EBRT. Additionally, there is a good agreement between both models, since the corresponding fit curves and 95% confidence intervals substantially coincide.

According to two-sided Wilcoxon rank-sum tests, the differences in quality of fit between the relapsing and the non-relapsing subgroups are significant for both the periodic dose model (RMSE: p<0.001, R²: p=0.0033) and the single dose model (RMSE: p<0.001, R²: p=0.0027). Corresponding one-sided tests show that superior fits are achieved for non-relapsing patients in terms of significantly lower RMSE and higher R² with both the periodic dose model (RMSE: p<0.001, R²: p=0.0017) and the single dose model (RMSE: p<0.001, R²: p=0.0013). On a patient-wise basis, Wilcoxon signed-rank testing shows that the single dose model outperforms the periodic dose model in the non-relapsing subgroup, yielding significantly lower RMSE and higher R² (p<0.001 for both metrics in two- and one-sided tests). However, these results do not hold in the subgroup of relapsing patients. Additionally, no significant global differences in the RMSE and R² distributions obtained using the periodic versus the single dose model are detected in any two-sided Wilcoxon rank-sum test performed across the whole cohort, the non-relapsing subgroup, and the relapsing one.

Mechanistic models provide promising biomarkers of biochemical relapse. Following global fitting, a panel of model-based quantities of interest were defined to examine their performance as biomarkers of biochemical relapse. As in Lorenzo et al. (2019), this panel is composed by three groups of quantities. The following model parameters were included: the baseline PSA (P₀), the proliferation rate of tumor cells (ρₛ), the rate of EBRT-induced death of tumor cells (ρ_{d}), and the fraction of tumor cells surviving to EBRT (R; R=R_{d} in the periodic dose model, R=R_{D} in the single dose model, and statistical analyses test embedded image versus R_{D}; see STAR Methods). Second, two non-dimensional metrics were considered that represent the ratio or tumor cell proliferation to EBRT-induced death (β) and EBRT efficacy (α). Finally, the models were used to calculate the PSA nadir (ρₙ) and the time to PSA nadir since EBRT termination (Δtₙ), which are two common metrics in the analysis of post-EBRT PSA dynamics. The interested reader is referred to the STAR Methods for further information on the definition of these model-based quantities of interest.

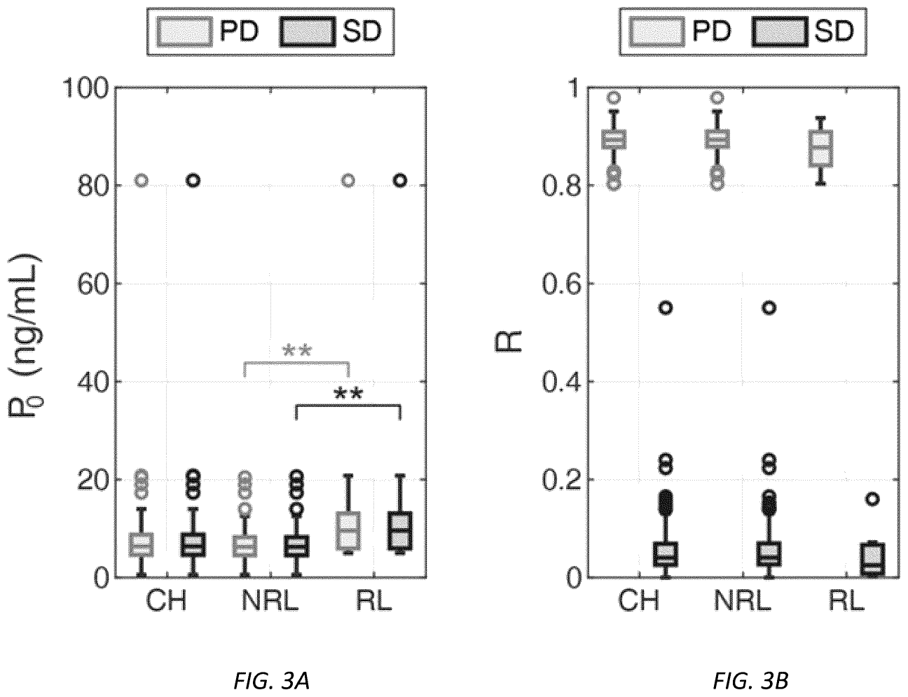

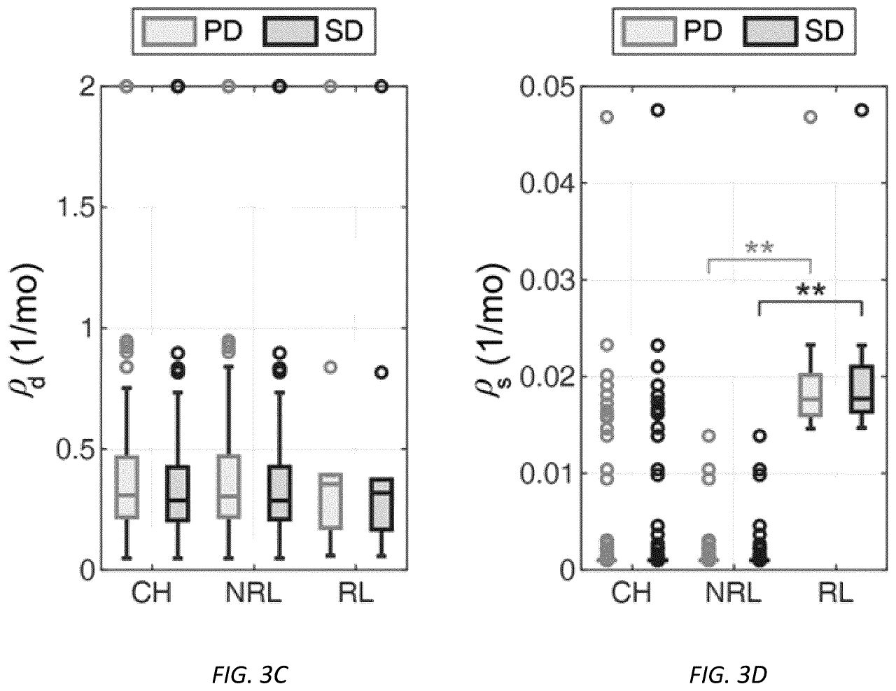

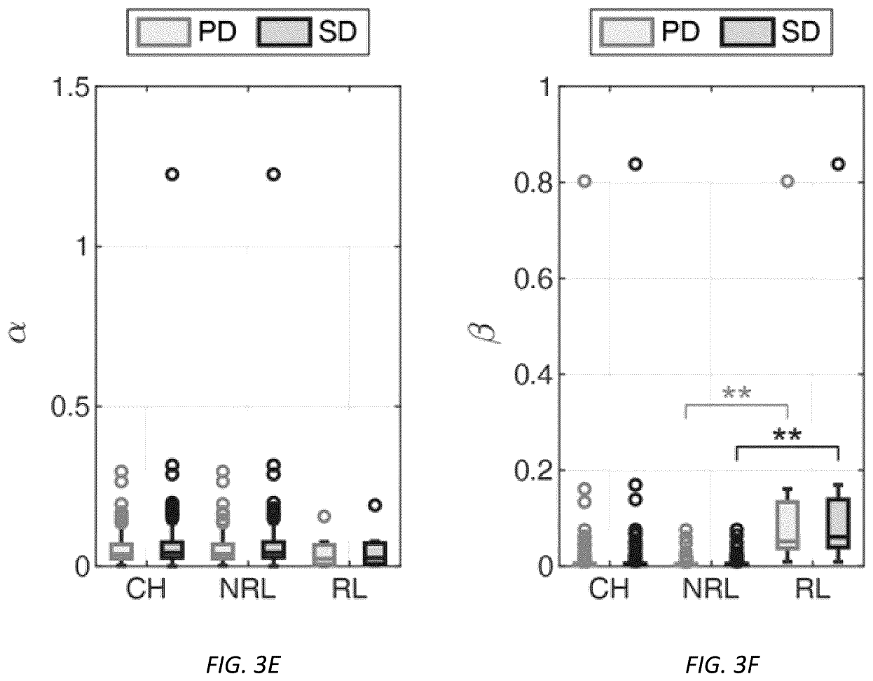

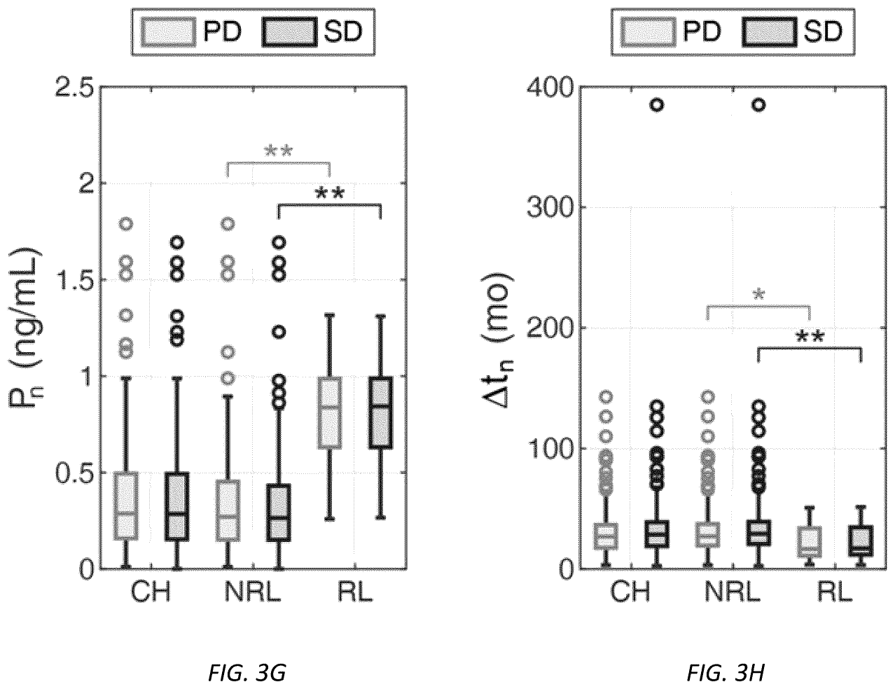

FIGS. 3A through 3H show the boxplots of the distributions of all model-based quantities of interest across the cohort as well as the subgroups of non-relapsing and relapsing patients. Two-sided Wilcoxon rank-sum tests identify significant differences in the values obtained for P₀, ρₛ, β, and ρₙ using either the periodic dose model (p=0.011, <0.001, <0.001, and <0.001, respectively) or the single dose model (p=0.0098, <0.001, <0.001, and <0.001, respectively). Corresponding one-sided tests further show that relapsing patients exhibit higher P₀, ρₛ, β, and ρₙ than non-relapsing patients utilizing either the periodic dose model (p=0.0053, <0.001, <0.001, and <0.001, respectively) or the single dose model (p=0.0049, <0.001, <0.001, and <0.001, respectively). Additionally, a two-sided Wilcoxon rank-sum test identified significant differences in Δtₙ calculated with the single dose model (p=0.040), but not in those obtained with the periodic dose model (p=0.053). However, corresponding one-sided tests resulted in significantly lower values of Δtₙ for relapsing patients using either the periodic dose model (p=0.027) or the single dose model (p=0.020). As a result of this statistical analysis, ρₛ, β, ρₙ, and Δtₙ were defined as model-based biomarkers of biochemical relapse. P₀ was not considered for two reasons. First, the baseline PSA measured in the clinic and reported in Table 1 (P_{d}) was already significantly higher in relapsing patients (two-sided Wilcoxon rank-sum test p=0.017). Additionally, P₀ is bound to take values close to P_{d} as result of model fitting (see FIG. 1 and Lorenzo et al. (2019)), thereby providing a limited contribution to the sequential patient-specific predictions of PSA dynamics and biochemical relapse obtained during the fitting-forecasting study.

Each of FIGS. 3A through 3H shows the boxplots corresponding to the distribution of a model-based quantity of interest over the whole cohort (CH), the non-relapsing subgroup (NRL), and the relapsing one (RL). Green boxplots show results from the periodic dose model (PD), while blue boxplots correspond to the single dose model (SD). Outliers are represented as hollow circles. A double asterisk (**) indicates statistical significance in both two-sided and one-sided Wilcoxon rank-sum tests, while a single asterisk (*) denotes statistical significance only under a one-sided Wilcoxon rank-sum test (p<0.05). (A) shows initial PSA, P₀. (B) shows fraction of tumor cells surviving to EBRT (R=R_{d} in the PD model, R=R_{D} in the SD model, and R_{d}ⁿ^{_{d}} was tested versus R_{D}; see STAR Methods). (C) shows rate of EBRT-induced death of tumor cells, ρ_{d}. (D) shows proliferation rate of tumor cells, ρₛ. (E) shows non-dimensional ratio α, representing EBRT efficiency (see STAR Methods). (F) shows non-dimensional ratio β, representing the ratio of tumor cell proliferation to EBRT-induced death (see STAR Methods). (G) shows PSA nadir, ρₙ. (H) shows time to PSA nadir since EBRT termination, Δtₙ.

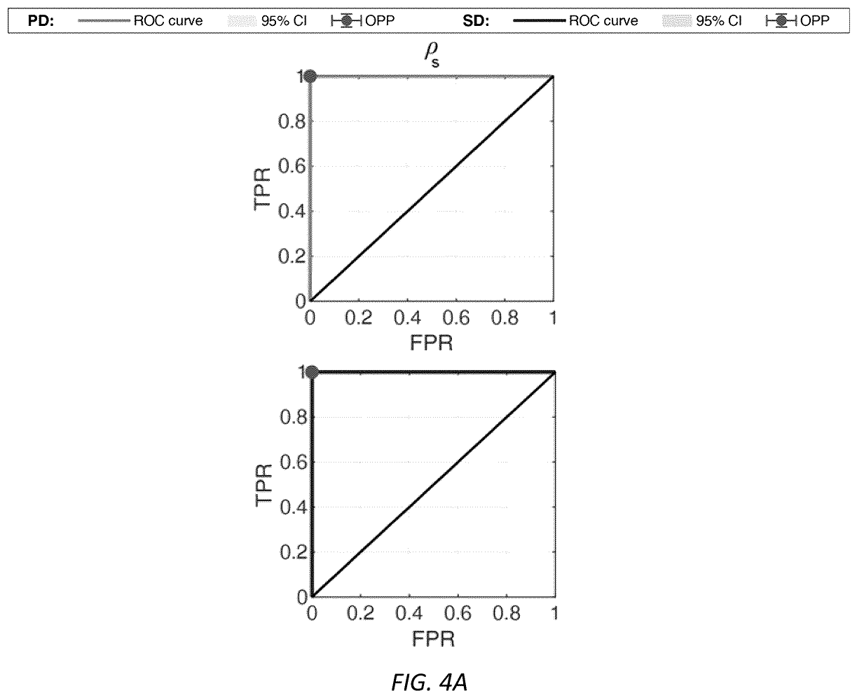

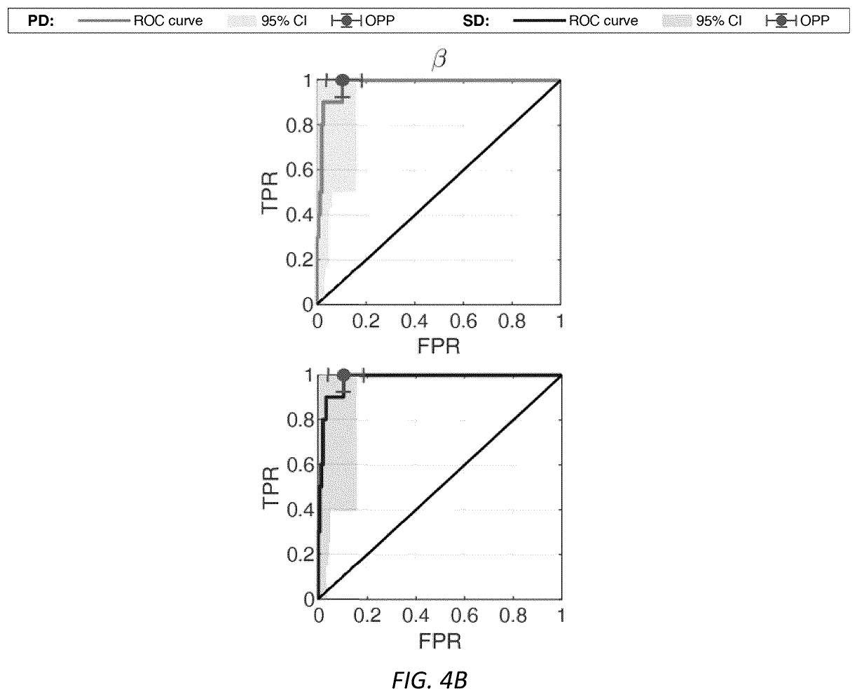

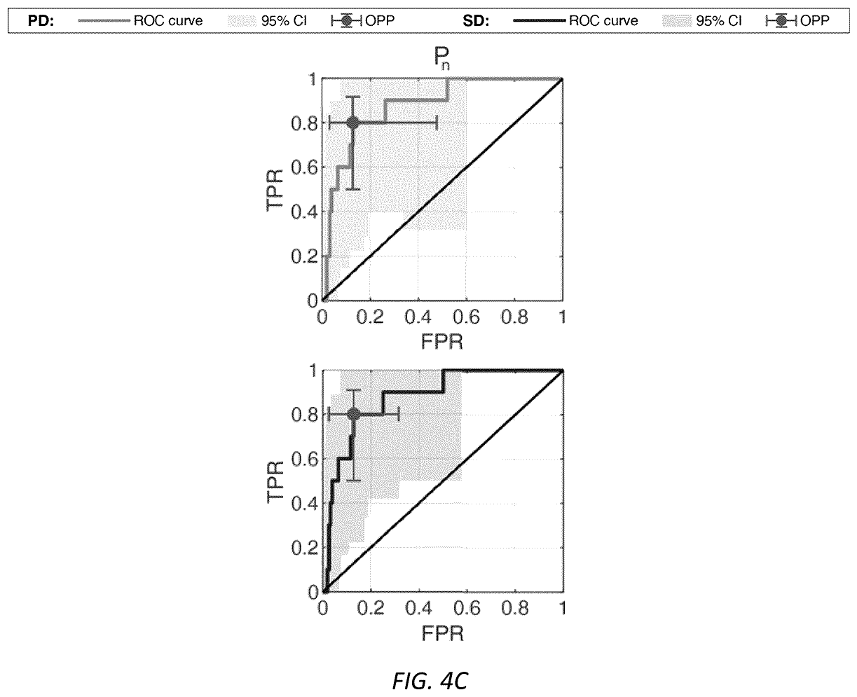

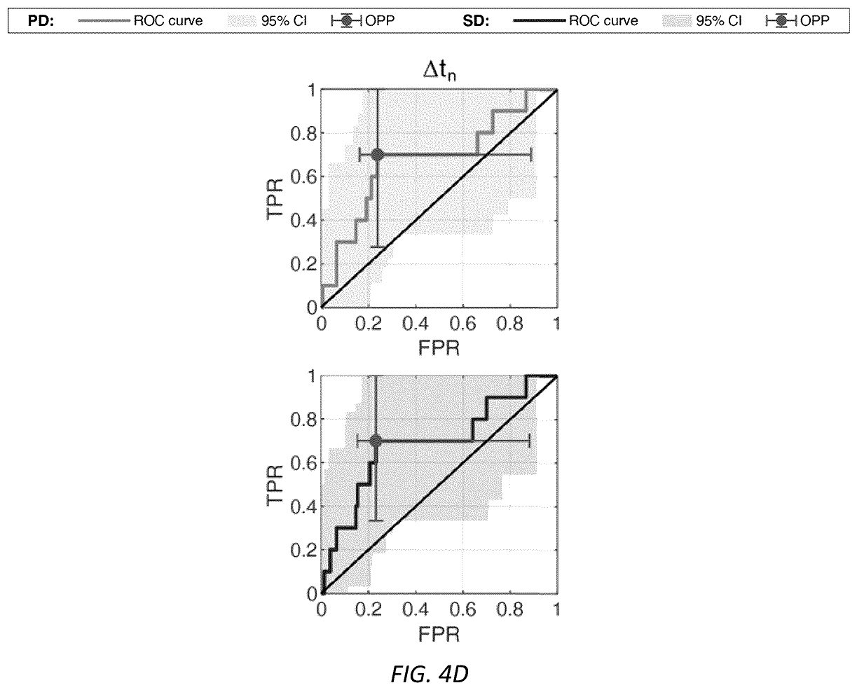

FIGS. 4A through 4D show the receiver operating characteristic (ROC) curves and corresponding optimal performance points for the model-based biomarkers identified from the analysis of the global fitting study results. FIGS. 4A-4D show the ROC curves and 95% bootstrap confidence intervals (CI) for each biomarker constructed using the global fitting results from the periodic dose model (PD, top row, in green) single dose model (SD, bottom row, in blue). In each plot, the vertical axis quantifies the true positive rate (TPR, i.e., sensitivity), while the horizontal axis measures the false positive rate (FPR, i.e., 1-specificity). The solid black line represents the unity line. The optimal performance point (OPP) and corresponding 95% bootstrap confidence intervals along both axes are depicted as a red solid point and error bars, respectively. (A) shows proliferation rate of tumor cells, ρₛ. (B) shows non-dimensional ratio β, representing the ratio of tumor cell proliferation to EBRT-induced death (see STAR Methods). (C) shows PSA nadir, Pₙ. (D) shows time to PSA nadir since EBRT termination, Δtₙ. For each biomarker and model, Table 2 further provides the area under the ROC curve (AUC) along with the optimal performance point threshold, sensitivity, and specificity. Specifically, Table 2 provides analysis of the ROC curves of the model-based biomarkers of biochemical relapse identified in the global fitting study. Values in brackets are 95% bootstrap confidence intervals of the reported metrics. AUC: area under the ROC curve. It was observed that the ROC curves and optimal performance points obtained for each biomarker with either model were very similar, which suggests that the biomarker performance is independent of the model leveraged in its calculation. The shape of the ROC curves, the AUC, and the optimal performance points show that ρₛ exhibited the best performance in identifying relapsing patients, followed closely by β, then Pₙ, and finally Δtₙ, which only shows a mildly satisfactory performance. It can be seen that ρₛ operates as a perfect classifier, yielding maximal AUC along with 100% sensitivity and specificity at optimal performance point. This result can also be hinted in the boxplots of this biomarker shown in FIGS. 3A through 3H, where a straight line could separate the values of ρₛ obtained with both models for relapsing and non-relapsing patients.

TABLE 2

| | Biomarker | | | |
|---|---|---|---|---|
| Metric | ρₛ | β | Pₙ | Δtₙ |
| Periodic dose model | | | | |
| AUC | 1.00 [1.00, 1.00] | 0.98 [0.93, 1.00] | 0.88 [0.68, 0.95] | 0.68 [0.45, 0.85] |
| Optimal performance point | | | | |
| Sensitivity | 1.00 [1.00, 1.00] | 1.00 [0.92, 1.00] | 0.80 [0.50, 0.92] | 0.70 [0.28, 1.00] |
| Specificity | 1.00 [1.00, 1.00] | 0.90 [0.82, 0.96] | 0.87 [0.52, 0.97] | 0.76 [0.11, 0.84] |
| Value | 14.6 [13.9, 14.6] · 10⁻³/mo | 9.0 [6.7, 22.8] · 10⁻³ | 0.6 [0.3, 0.9] ng/mL | 19.0 [15.9, 53.6] mo |
| Single dose model | | | | |
| AUC | 1.00 [1.00, 1.00] | 0.98 [0.94, 1.00] | 0.88 [0.71, 0.95] | 0.69 [0.47, 0.87] |
| Optimal performance point | | | | |
| Sensitivity | 1.00 [1.00, 1.00] | 1.00 [0.92, 1.00] | 0.80 [0.50, 0.91] | 0.70 [0.33, 1.00] |
| Specificity | 1.00 [1.00, 1.00] | 0.90 [0.82, 0.96] | 0.87 [0.68, 0.97] | 0.77 [0.12, 0.85] |
| Value | 14.7 [13.9, 14.7] · 10⁻³/mo | 9.0 [7.6, 23.4] · 10⁻³ | 0.6 [0.4, 0.9] ng/mL | 19.8 [16.2, 54.7] mo |

Mechanistic models accurately forecast short-term PSA dynamics after EBRT. To assess the predictive potential of the model-based biomarkers of biochemical relapse identified in the global fitting study, first the accuracy of the predictions of PSA dynamics obtained with the mechanistic models was analyze. To this end, a fitting-forecasting study was run as follows: first, the models' parameters are estimated by fitting them to a subset of the earliest PSA values for each patient. Then, the corresponding personalized models' forecasts of PSA dynamics is calculated. Finally, each of these predictions is compared to the remainder of the patient's PSA data in posterior dates. For each patient, this calculation is performed in a collection of sequential scenarios: start using a minimum of five PSA values for fitting (n_{P,fit}=5) including the baseline PSA at diagnosis (P_{d}), and progressively increase n_{P,fit} until only one PSA value is left for assessing the models' predictions (i.e., n_{P,fit}=5, . . . , n_{P-1} for each patient; see STAR Methods for further methodological details). Additionally, this study considers a maximum n_{P,fit}=21, since this is the last scenario for which there is at least one relapsing and one non-relapsing patient with at least one remaining PSA value to assess the models' forecasts.

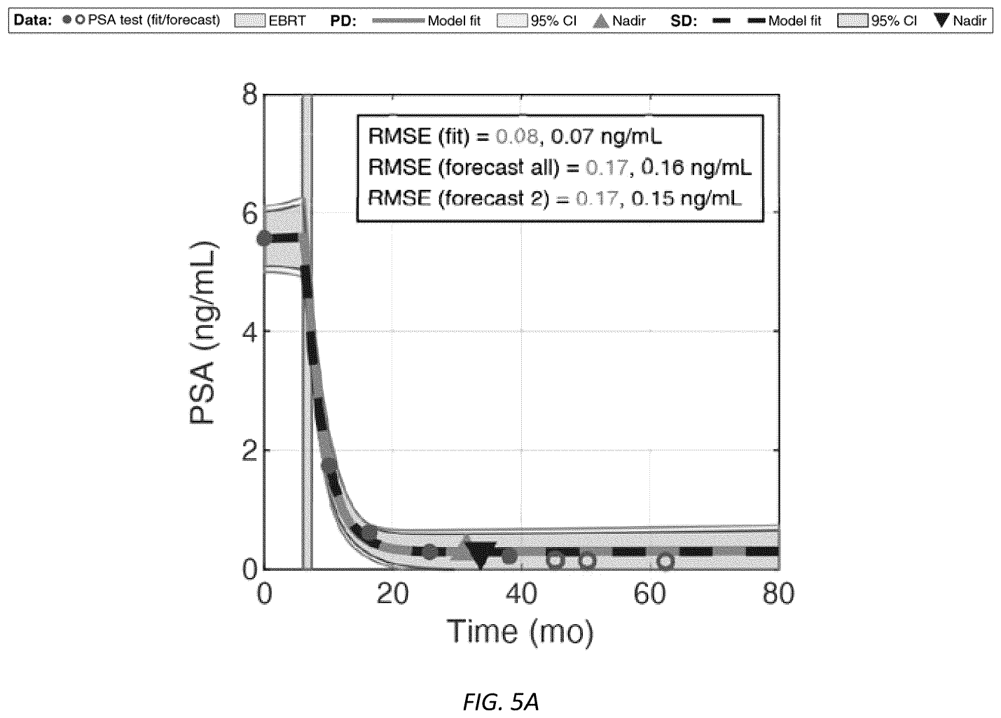

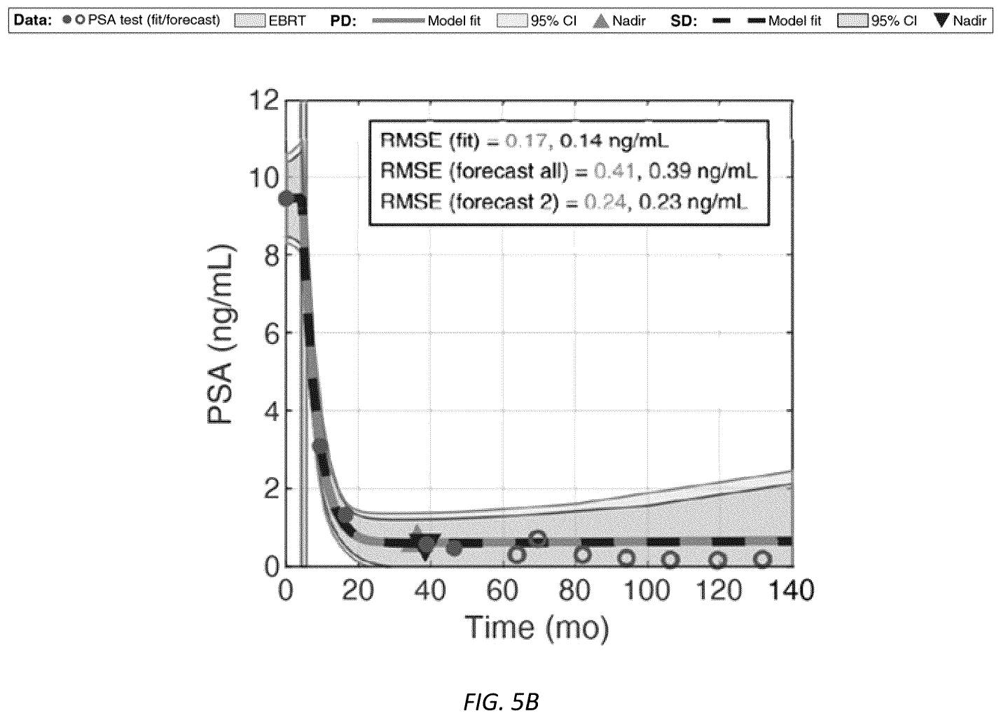

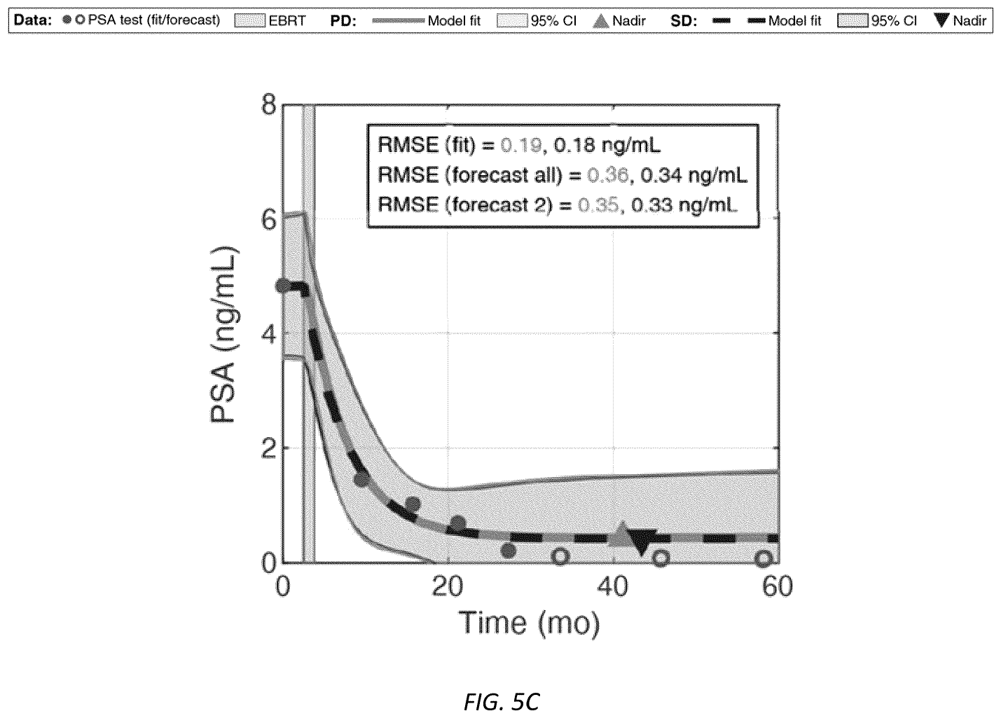

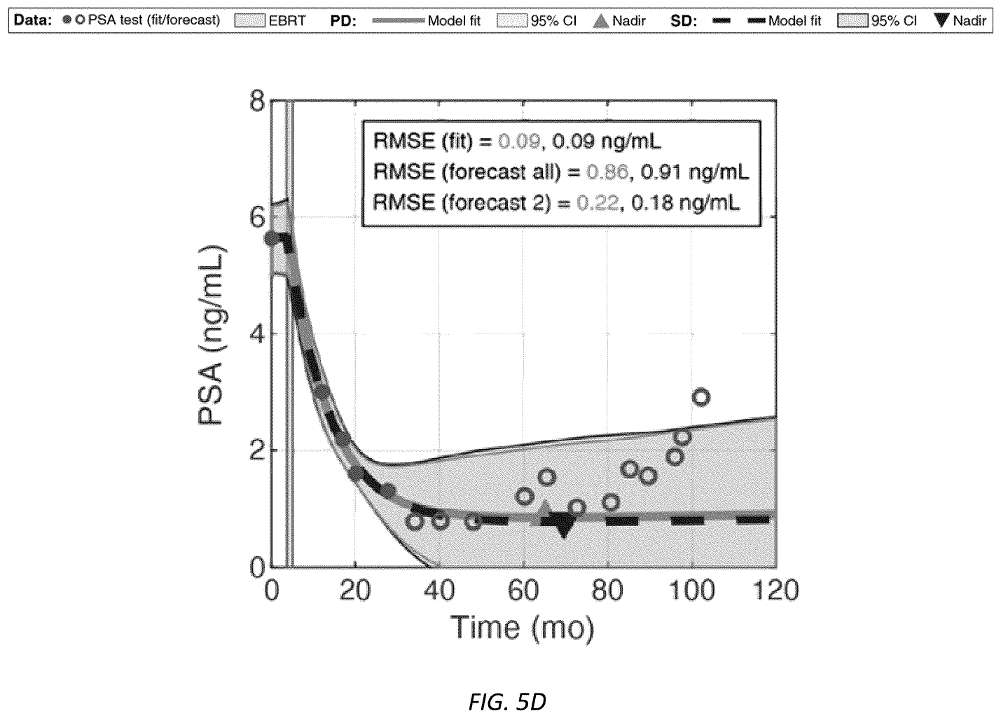

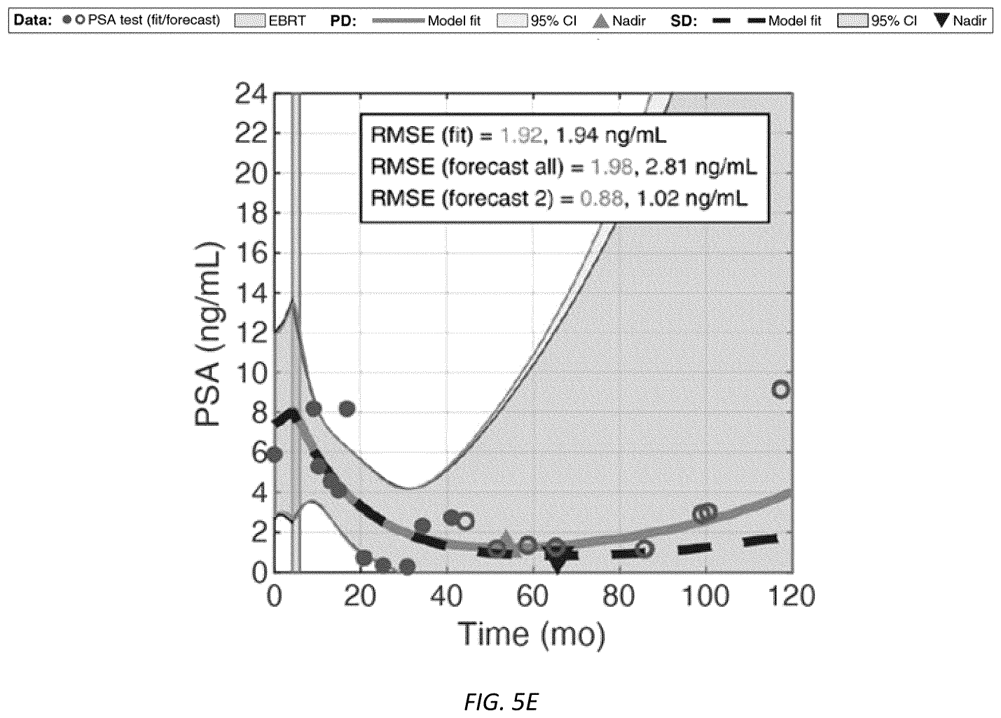

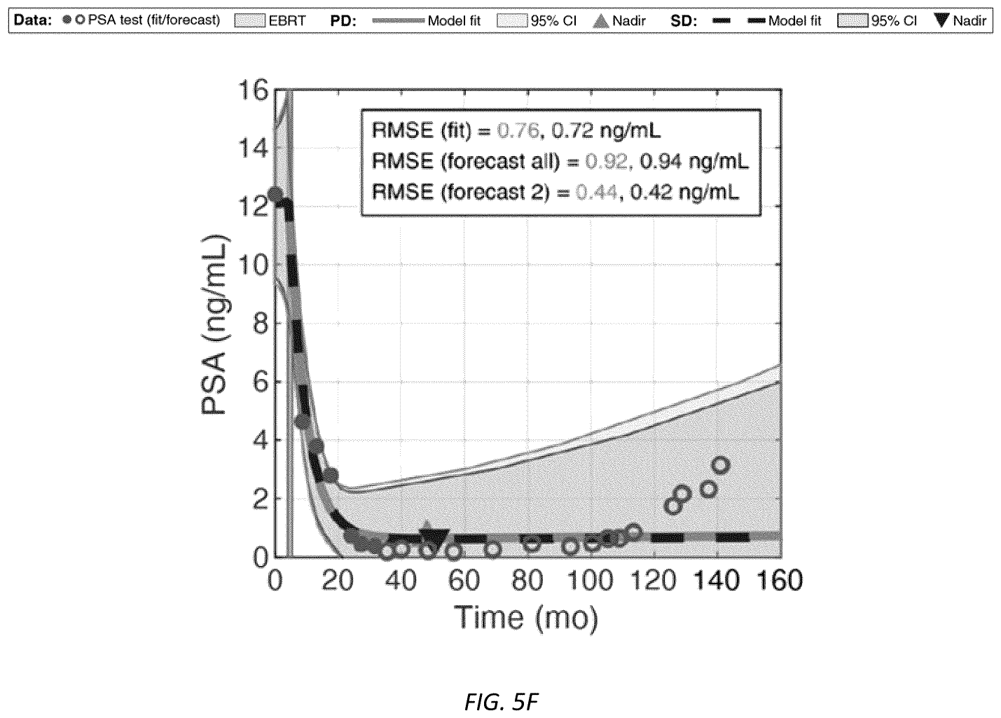

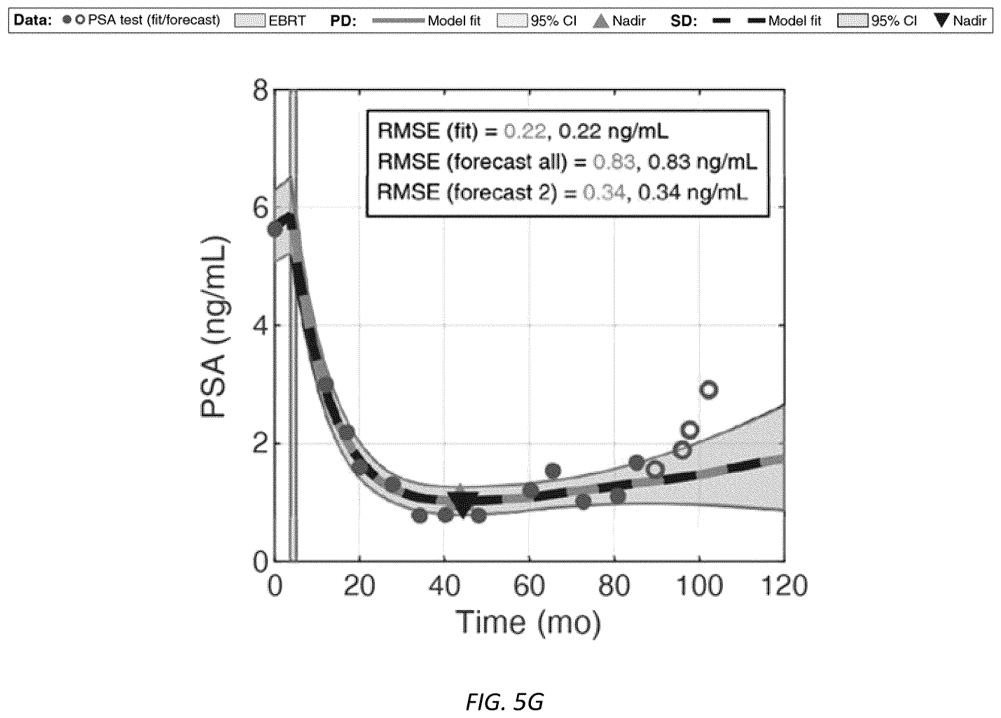

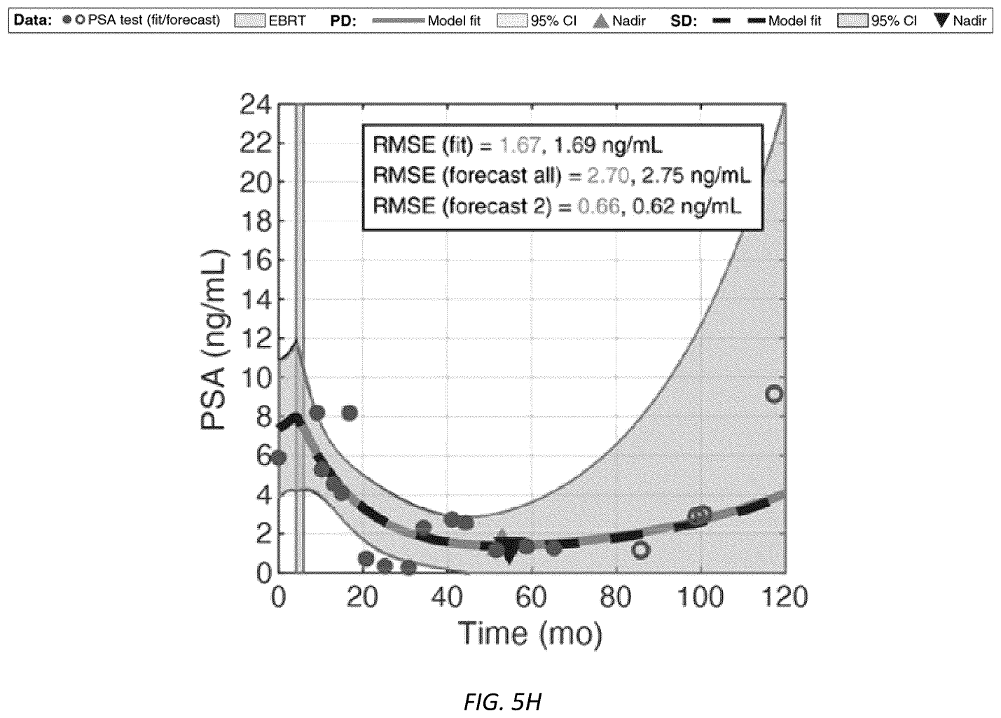

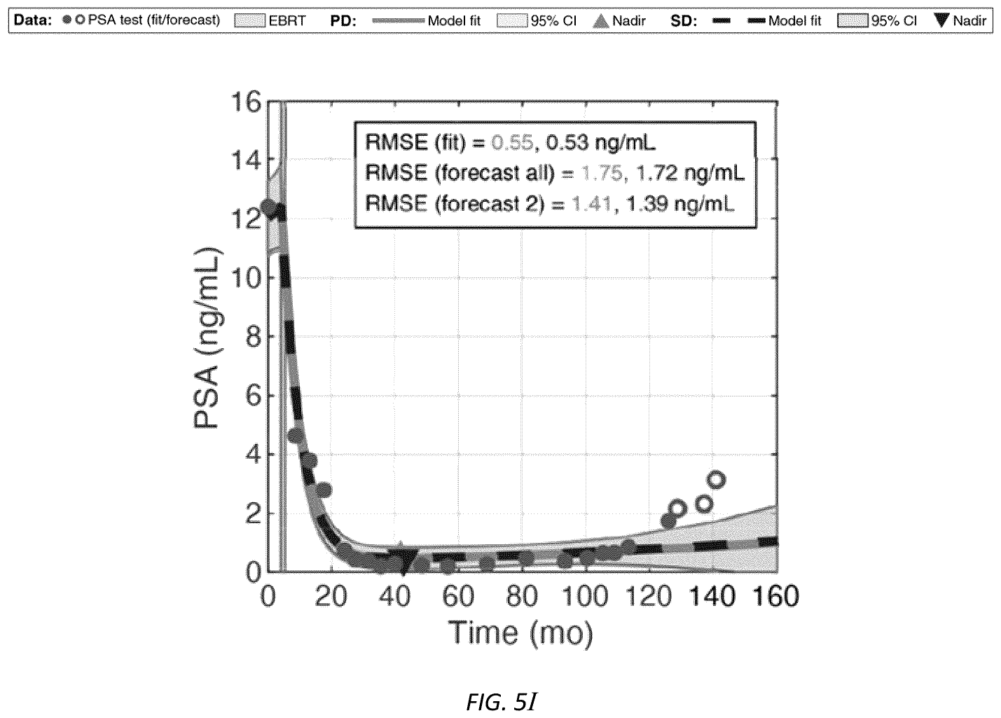

FIGS. 5A through 5I illustrate representative results from the fitting-forecasting study for the relapsing and non-relapsing patients considered in FIGS. 2A through 2F in the global fitting study. FIGS. A-C and FIGS. D-F show an early forecast of the PSA dynamics obtained with the mechanistic models for the same non-relapsing and relapsing patients considered in FIGS. 2A through 2F, respectively. The early model forecasts for the non-relapsing patients accurately predict both short and long-term PSA dynamics. However, the early forecasts of PSA are only accurate in the short term for the relapsing patients. FIGS. G-I show the model forecasts of PSA dynamics obtained at a later date in the course of post-EBRT monitoring for the same non-relapsing patients (i.e., in a higher n_{P,fit} scenario). In this case, while the models can still predict only the following short-term PSA values accurately, they can already detect a rising PSA trend earlier than standard clinical practice (e.g., using the nadir+2 ng/ml criterion). Moreover, the PSA predictions and corresponding 95% confidence intervals in this figure demonstrate an overall good agreement between the models in forecasting PSA dynamics. For each patient, the PSA measurements used for model fitting are plotted as red bullet points, while the remainder PSA data to assess model predictions are represented as hollow red circles. EBRT is represented as a light gray rectangular area. The fits and forecasts obtained with the periodic dose model (PD) are represented with a solid green line, while the corresponding results produced by the single dose model (SD) are depicted with a dashed blue line. The same color scheme is also used for the 95% confidence intervals, which are depicted as shaded areas around the model fits, and the font of the fitting and forecasting RMSEs for either model. The forecasting RMSE is reported for both all the remaining PSA values and the immediately next two PSA values after the date of the last PSA used for model fitting. Additionally, each plot also includes the prediction of the patient's PSA nadir using the periodic dose model (green upward triangle) and the single dose model (blue downward triangle).

The results represented in FIG. 5 show that these models can accurately predict PSA in the short term after the date of the last PSA used in model fitting. This is the case for both non-relapsing and relapsing patients across the different n_{P,fit} scenarios considered in the analysis. While the prediction of the long-term PSA plateau in the non-relapsing subgroup is accurate even with a low n_{P,fit} (see FIGS. 5A-5C), the models may require exposition to an incipient rising trend to accurately identify a biochemical relapse and estimate long-term PSA values (see FIGS. 5D-5F versus FIGS. 5G-5I). However, FIGS. 5G-5I further show that the model forecasts are able to identify rising PSA dynamics in relapsing patients earlier than standard clinical detection methods (e.g., using the nadir+2 ng/ml criterion). Additionally, as in the global fitting study, it can be observed that an overall good agreement between the fitting and forecasting results obtained with the periodic dose and the single dose models. Comparing FIGS. 5A-5I and FIGS. 2A-2F, it can be observed that, as the number of PSA values used for model fitting increases (i.e., for higher n_{P,fit}), the uncertainty in the model predictions of PSA decreases accordingly and progressively approaches the level of uncertainty obtained in the global fitting scenario.

To analyze the distribution of the global RMSE values across all the fitting and forecasting scenarios, the corresponding results obtained with either model for all n_{P,fit} cases run for each patient in the cohort are pooled. Since the mechanistic models provide an accurate prediction of short-term dynamics in both non-relapsing and relapsing patients, the analysis of the forecasting results focuses on the subsequent two PSA values that were not used in model fitting in each n_{P,fit} scenario for each patient. Hence, this two-value forecast corresponds to a short-term prediction of PSA dynamics over approximately a one-year horizon according to the PSA test frequency in the cohort (see Table 1). Note that not all patients for which it is possible to fit n_{P,fit} PSA values are eligible for a short-term prediction of two PSA values, because of limited PSA data for forecast validation.

Using the periodic dose model, the median and interquartile range of the RMSE of the model fits are 0.16 (0.08, 0.25) ng/mL across the whole cohort (n=1043), 0.15 (0.08, 0.23) ng/ml in the non-relapsing subgroup (n=949), and 0.34 (0.16, 0.76) ng/mL in the relapsing subgroup (n=94). For the short-term prediction of PSA using the patient-specific periodic dose model fits, the corresponding median and interquartile range of the RMSE are 0.18 (0.08, 0.36) ng/mL across the whole cohort (n=878), 0.17 (0.07, 0.32) ng/mL in the non-relapsing subgroup (n=794), and 0.67 (0.22, 1.20) ng/ml in the relapsing subgroup (n=84). Furthermore, the median and interquartile range of the RMSE of the single dose model fits are 0.15 (0.08, 0.25) ng/mL across the whole cohort (n=1043), 0.14 (0.07, 0.22) ng/mL in the non-relapsing sub-group (n=949), and 0.34 (0.17, 0.72) ng/mL in the relapsing subgroup (n=94). Using the patient-specific single dose model fits to perform a short-term forecast of PSA, the corresponding median and interquartile range of the RMSE are 0.18 (0.08, 0.34) ng/ml across the whole cohort (n=878), 0.16 (0.07, 0.31) ng/mL in the non-relapsing subgroup (n=794), and 0.63 (0.22, 1.19) ng/ml in the relapsing subgroup (n=84). These RMSE results demonstrate that the models can reproduce the observed PSA dynamics, and that the resulting personalized models can yield a reasonably accurate prediction of PSA to inform the monitoring strategy for each patient (see Discussion).

Considering the pooled results across all n_{P,fit} cases, the RMSE values of the model fits and the short-term PSA predictions obtained for non-relapsing patients were significantly lower than those obtained for relapsing patients. This happens using both the periodic dose and the single dose model (p<0.001 in all corresponding two- and one-sided Wilcoxon rank-sum tests). On a patient-wise basis, Wilcoxon signed-rank testing shows that the single dose model rendered significantly lower RMSE values than the periodic dose model during both fitting and short-term forecasting in the non-relapsing subgroup (p<0.001 for both two- and one-sided tests). However, this result does not hold in the relapsing subgroup. Furthermore, two-sided Wilcoxon rank-sum tests do not detect significant differences between the distributions of the global RMSE values of the model fits obtained with either model across the whole cohort, the non-relapsing subgroup, and the relapsing subgroup. The same result is also obtained when comparing the short-term PSA forecasts obtained with either model. Using two-sided Wilcoxon rank-sum test to compare the global RMSE distributions obtained for model fits against those calculated for the short-term PSA predictions, significant differences across the whole cohort, the non-relapsing subgroup, and the relapsing subgroup for both the periodic dose model (p<0.001, p<0.001, and p=0.018, respectively) and the single dose model (p<0.001, p<0.001, and p=0.025, respectively) are obtained. Corresponding one-sided tests further confirm that the RMSE values for the model fits are significantly lower than those of the short-term PSA predictions for both the periodic dose model (p<0.001, p<0.001, and p=0.0092, respectively) and the single dose model (p<0.001, p<0.001, and p=0.013, respectively).

Early estimates of model-based biomarkers obtained according to the methods and systems disclosed herein were found to accurately predict biochemical relapse earlier than standard practice. The patient-specific model fits obtained for each n_{P,fit} scenario in the fitting-forecasting study provide a set of values for the model-based biomarkers of biochemical relapse identified during global fitting (i.e., ρₛ, β, ρₙ, and Δtₙ). Since each n_{P,fit} scenario can only be performed once the latest PSA value used for model fitting is collected, the corresponding estimates of the biomarkers are associated with its collection date. Thus, it can be analyzed whether early estimates of the model-based biomarkers enable an accurate identification of relapsing patients and whether they anticipate the detection of relapse with respect to standard clinical practice. As further motivation for this analysis, for the three relapsing patients shown in FIGS. 5G-5I, the models predict a rising PSA trend before the usual nadir+2 ng/ml criterion is satisfied.

To assess the performance of the early model-based biomarker estimates as biochemical relapse classifiers, a ROC curve analysis is performed. All the values obtained for each biomarker in all the n_{P,fit} scenarios across all patients are pooled. Then, the ROC curve is constructed by using each pooled biomarker value as a threshold that is compared to the corresponding n_{P,fit} values obtained for each patient in the fitting-forecasting study (n_{P,fit}=5, . . . , n_{P-1}). If any of these patient-specific n_{P,fit} values satisfy the classification criterion for the biomarker (i.e., a larger ρₛ, β, and ρₙ or a lower Δtₙ), then the patient is identified as relapsing under the considered threshold. As in the global fitting study, this ROC curve analysis is performed for each model separately in accordance with the STAR methods.

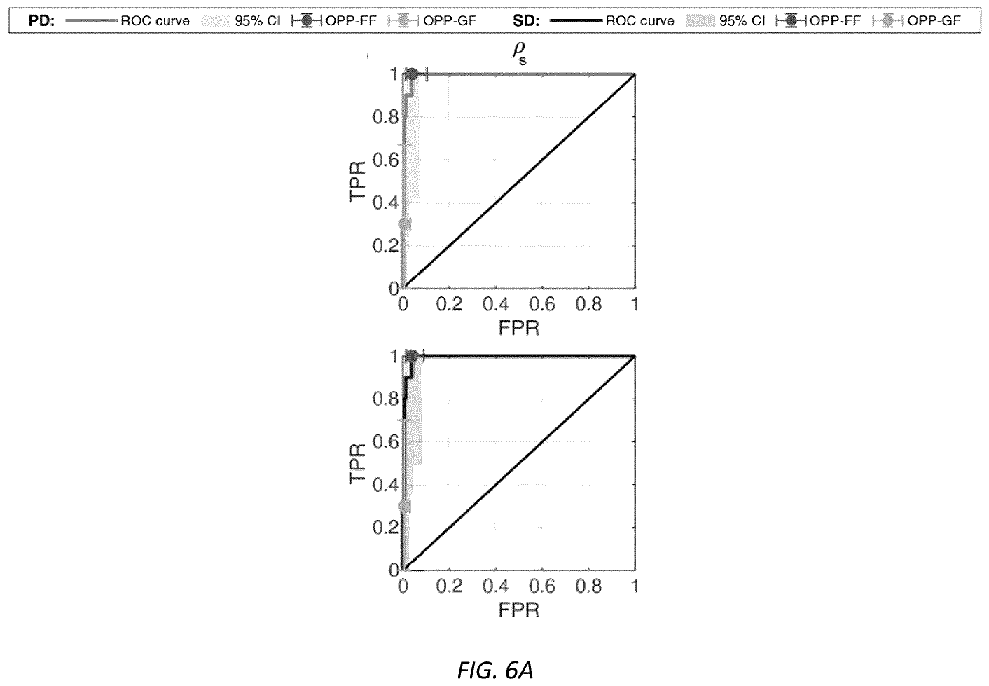

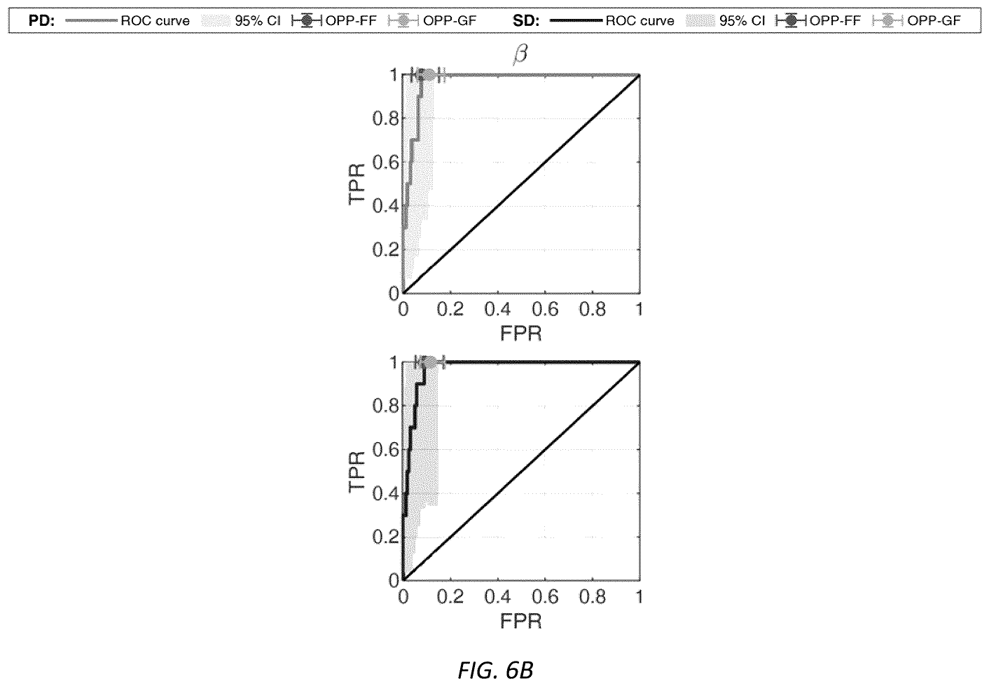

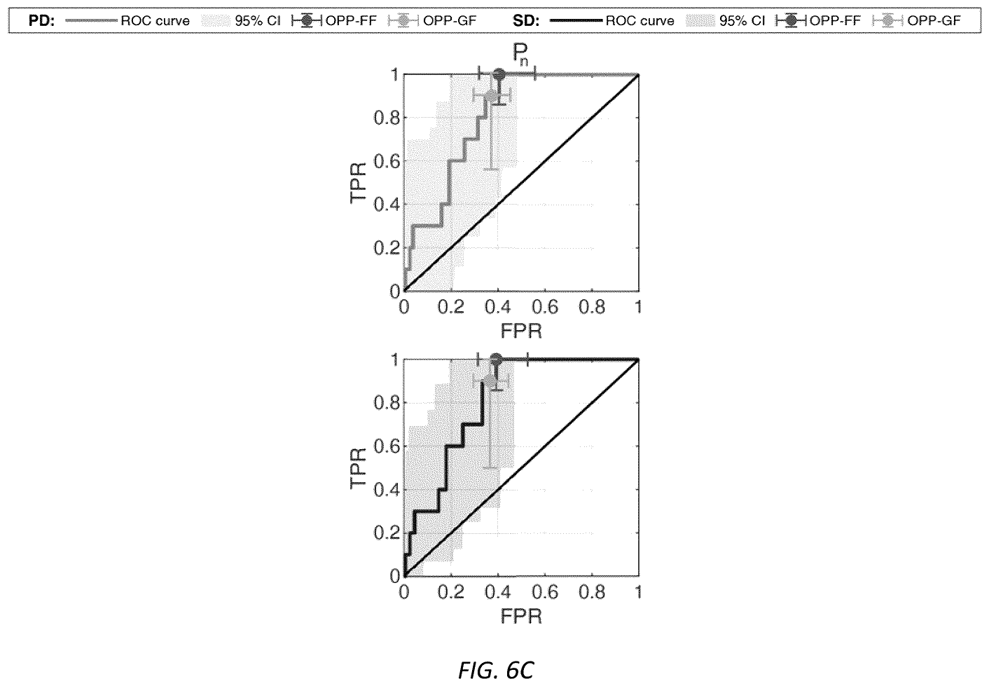

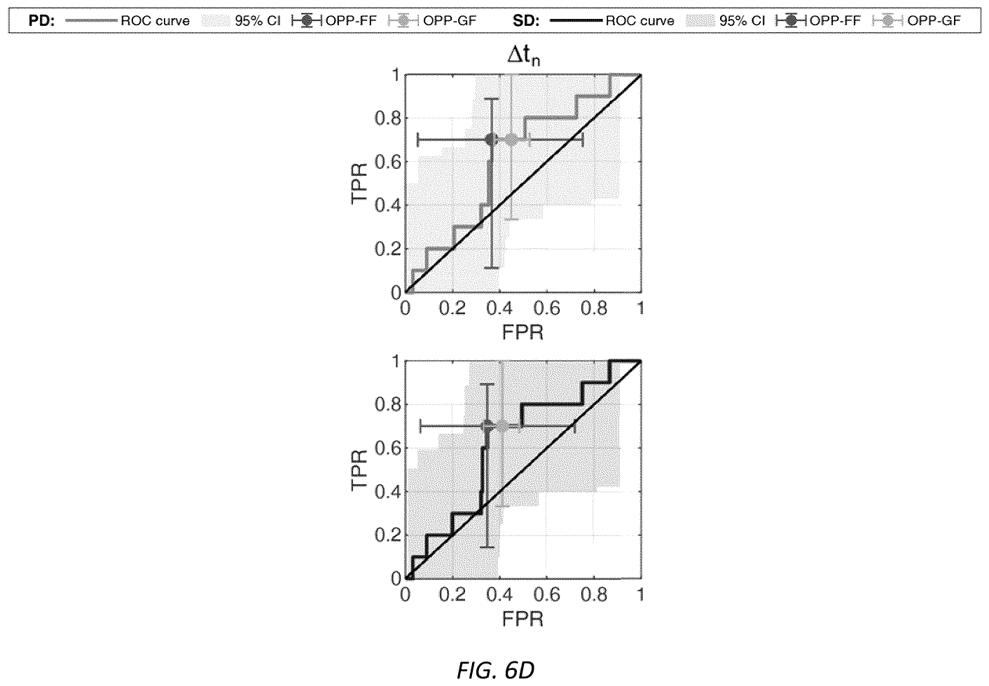

FIGS. 6A through 6D show the ROC curve and optimal performance point for each biomarker obtained by using both the periodic and the single dose model results of the fitting-forecasting study. FIGS. 6A-6D show the ROC curves and 95% bootstrap confidence intervals (CI) for each biomarker constructed using the fitting-forecasting results from the periodic dose model (PD, top row, in green) single dose model (SD, bottom row, in blue). In each plot, the vertical axis quantifies the true positive rate (TPR, i.e., sensitivity), while the horizontal axis measures the false positive rate (FPR, i.e., 1-specificity). The solid black line represents the unity line. The optimal performance point (OPP-FF) and corresponding 95% bootstrap confidence intervals along both axes are depicted as a red solid point and error bars, respectively. Additionally, the optimal performance point identified during global fitting (OPP-GF) and corresponding 95% bootstrap confidence intervals are represented with a pink solid point and error bars, respectively. (A) Proliferation rate of tumor cells, ρₛ. (B) Non-dimensional ratio β, representing the ratio of tumor cell proliferation to EBRT-induced death (see STAR Methods). (C) PSA nadir, Pₙ. (D) Time to PSA nadir since EBRT termination, Δtₙ.

Table 3 further reports the corresponding AUC value along with the optimal performance point threshold, sensitivity, and specificity. Table 3 shows analyses of the ROC curves of the model-based biomarkers to identify early biochemical relapse using the fitting-forecasting study results. Values in brackets are 95% bootstrap confidence intervals of the reported metrics. For each biomarker, the global fitting threshold was fixed to the optimal cutoff reported in Table 2 in order to determine the corresponding 95% bootstrap confidence intervals for its sensitivity and specificity using the fitting-forecasting study results. AUC: area under the ROC curve.

TABLE 3

| | Biomarker | | | |
|---|---|---|---|---|
| Metric | ρₛ | β | Pₙ | Δtₙ |
| Periodic dose model | | | | |
| AUC | 0.99 [0.97, 1.00] | 0.97 [0.93, 0.99] | 0.81 [0.71, 0.90] | 0.62 [0.42, 0.77] |
| Optimal performance point | | | | |
| Fining-forecasting study | | | | |
| Sensitivity | 1.00 [1.00, 1.00] | 1.00 [1.00, 1.00) | 1.00 [0.86, 1.00] | 0.70 [0.11, 0.89] |
| Specificity | 0.96 [0.90, 0.99] | 0.92 [0.85, 0.96] | 0.60 [0.45, 0.68] | 0.63 [0.25, 0.95] |
| Value | 5.6 [2.0, 9.1] · 10⁻³/mo | 13.0 [8.8, 16.4] · 10⁻³ | 0.6 [0.5, 0.7] ng/mL | 16.7 [8.3, 36.6] mo |
| Global fitting study | | | | |
| Sensitivity | 0.30 [0.00, 0.67] | 1.00 [1.00, 1.00] | 0.90 [0.57, 1.00] | 0.70 [0.33, 1.00] |
| Specificity | 0.99 [0.97, 1.00] | 0.89 [0.83, 0.94] | 0.63 [0.55, 0.70] | 0.55 [0.47, 0.62] |
| Value | 14.6 · 10⁻³ 1/mo | 9.0 · 10⁻³ | 0.6 ng/mL | 19.0 mo |
| Single dose model | | | | |
| AUC | 0.99 [0.97, 1.00] | 0.97 [0.93, 0.99] | 0.81 [0.70, 0.90] | 0.63 [0.44, 0.78] |
| Optimal performance point | | | | |
| Fining forecasting study | | | | |
| Sensitivity | 1.00 [1.00, 1.00] | 1.00 [1.00, 1.00] | 1.00 [0.86, 1.00] | 0.70 [0.14, 0.89] |
| Specificity | 0.96 [0.91, 0.99] | 0.91 [0.83, 0.95] | 0.61 [0.47, 0.69] | 0.65 [0.28, 0.94] |
| Value | 5.7 [2.4, 10.0] · 10⁻³/mo | 13.3 [7.6, 17.4] · 10⁻³ | 0.6 [0.6, 0.7] ng/mL | 18.1 [7.9, 39.6] mo |
| Global firing study | | | | |
| Sensitivity | 0.30 [0.00, 0.70] | 1.00 [1.00, 1.00] | 0.90 [0.50, 1.00] | 0.70 [0.33, 1.00] |
| Specificity | 0.99 [0.97, 1.00] | 0.88 [0.82, 0.93] | 0.63 [0.56, 0.71] | 0.59 [0.52, 0,67] |
| Value | 14.7 · 10⁻³ 1/mo | 9.0 · 10⁻³ | 0.6 ng/mL | 19.8 mo |

Additionally, both FIGS. 6A through 6D and Table 3 represent the ability of the optimal performance point threshold identified in the global fitting study to classify biochemical relapse using the early biomarker estimates from the fitting-forecasting study. The ROC curves and optimal performance points obtained with either model are virtually equivalent, as we had previously observed in the ROC curve analysis of the global fitting results (see FIGS. 4A-4D and Table 2). Furthermore, it is again seen that the best performing biomarkers are ρₛ and β, followed by Pₙ, and finally Δtₙ, which exhibits a comparably poorer classifying ability with respect to the other three biomarkers. Comparing the ROC curve metrics obtained in the global fitting study and the fitting-forecasting study, the AUC is slightly lower when the biomarkers obtained with either model are assessed to early identify biochemical relapse. While parameter ρₛ acted as a perfect classifier in the global fitting study, a minor loss of specificity is observed when it is leveraged as an early biomarker for biochemical relapse. In addition, the optimal threshold required to provide an early classification of biochemical relapse with ρₛ is notably lower with respect to the one obtained in the global fitting study. Thus, a less conservative threshold is needed to provide an early identification of biochemical relapse using ρₛ. This is also suggested by comparing the model predictions for the relapsing patients in FIGS. 2D-2F and FIGS. 5G-5I. Conversely, the β ratio exhibits a slightly better performance in the fitting-forecasting study due to a moderate increase in specificity, and the optimal threshold stays in the same order of magnitude. This last observation also holds for the PSA nadir Pₙ, which shows maximal sensitivity in the fitting-forecasting study. However, its specificity to early detect biochemical relapse is lower than in the global fitting study. Finally, Δtₙ also exhibits a lower specificity in the fitting-forecasting study than in the global fitting scenario.

The optimal threshold calculated for the ROC curve of each model-based biomarker in the fitting-forecasting study is leveraged to assess whether it may enable an earlier detection of biochemical relapse than standard clinical criteria (e.g., nadir+2 ng/ml). To this end, a metric termed “Days Gained to Biochemical Relapse Diagnosis” (DGBRD) is defined for each biomarker and for every patient in the relapsing subgroup. This metric is computed as the difference between the reported date of biochemical relapse and the earliest date in which each biomarker classifies a patient as relapsing according to the optimal threshold calculated in the ROC curve analysis of the fitting-forecasting study. A positive value of DGBRD indicates that a biomarker enables the early detection of biochemical relapse with respect to standard clinical practice. It can be seen that the second date in the DGBRD definition corresponds to one of the dates in which PSA was measured for each relapsing patient, since these are the dates at which we calculate the model fits in each of the n_{P,fit} scenarios of the fitting-forecasting study.

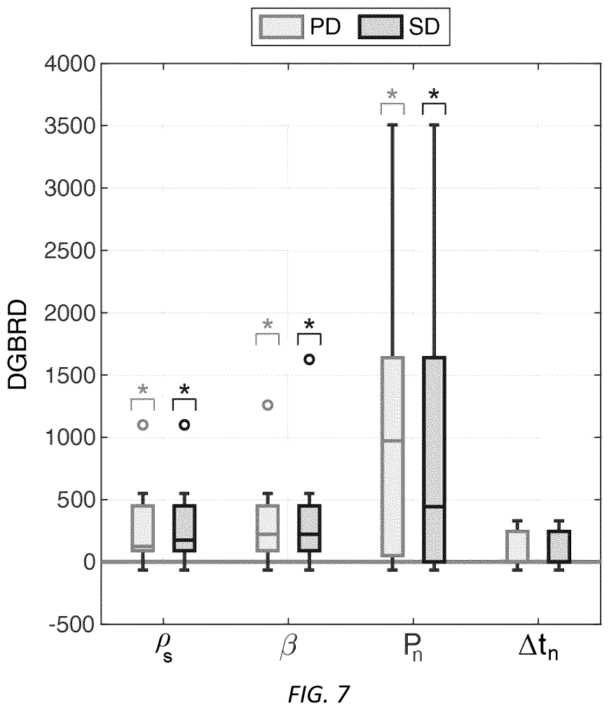

FIG. 7 shows the distribution of the DGBRD for each biomarker calculated with both the periodic and the single dose model. In FIG. 7, the boxplots in this figure represent the distribution of the DGBRD in the relapsing subgroup (n=10) obtained for each biomarker of biochemical relapse by leveraging the optimal threshold calculated in the ROC curve analysis of the fitting-forecasting study results for both the periodic dose and the single dose model. From left to right: proliferation rate of tumor cells ρₛ, non-dimensional ratio β (i.e., ratio of tumor cell proliferation to EBRT-induced death; see STAR Methods), PSA nadir Pₙ, and time to PSA nadir since EBRT termination Δtₙ. Green boxplots show results from the periodic dose model (PD), while blue boxplots correspond to the single dose model (SD). Outliers are represented as hollow circles. A single asterisk (*) denotes statistical significance in one-sided Wilcoxon signed-rank tests for a median larger than zero (p<0.05). Table 4 reports the corresponding median, interquartile range, and full range relative to distribution of the Days Gained to Biochemical Relapse Diagnosis (DGBRD) in the relapsing subgroup (n=10) for each biomarker obtained leveraging the optimal threshold calculated in the ROC curve analysis of the fitting-forecasting results for both the periodic dose and the single dose model. IQR: interquartile range.

TABLE 4

| | Periodic dose model | | | Single dose model | | |
|---|---|---|---|---|---|---|
| Biomarker | Median | IQR | Range | Median | IQR | Range |
| ρₛ | 125 | (90, 450) | (−66, 1100) | 175 | (90, 450) | (−66, 1100) |
| β | 222 | (90, 450) | (−66, 1259) | 222 | (90, 450) | (−66, 1625) |
| Pₙ | 971.5 | (51, 1639) | (−66, 3505) | 443.5 | (0, 1639) | (−66, 3505) |
| Δtₙ | 0 | (0, 245) | (−66, 330) | 0 | (0, 245) | (−66, 330) |

Using one-sided signed-rank Wilcoxon tests on median larger than zero, the early estimates of ρₛ, β, and Pₙ provide a significantly earlier detection of biochemical relapse than standard clinical practice either leveraging the periodic dose model (p=0.0029, 0.0029, and 0.0056, respectively) or the single dose model (p=0.0029, 0.0029, and 0.0098, respectively) for their calculation. For both models, while ρₛ and β exhibited the best classifier performance in the ROC curve analysis (see FIGS. 5A-5I and Table 3), Pₙ is the biomarker yielding the earliest identification of biochemical relapse. Indeed, two-sided Wilcoxon signed-rank tests identify significant patient-wise differences in the DGBRD for Pₙ with respect to those calculated for ρₛ, β, and Δtₙ for both the periodic dose model (p=0.016, 0.016, and 0.0078, respectively) and the single dose model (p=0.019, 0.019, and 0.016, respectively). Corresponding one-sided tests confirm that the DGBRD calculated for Pₙ provided a significantly earlier identification of biochemical relapse than the DGBRD obtained for ρₛ, β, and Δtₙ for each patient with both the periodic dose model (p=0.0078, 0.0078, and 0.0039, respectively) and the single dose model (p=0.0098, 0.0098, and 0.0078, respectively). The comparison of the DGBRD for ρₛ and β with respect to the DGBRD for Δtₙ from the single dose model was not significant under two-sided Wilcoxon signed-rank testing. However, the corresponding one-sided tests do identify a significantly larger DGBRD for ρₛ and β (p=0.039 and p=0.027, respectively). For the periodic dose model, this observation only holds for the comparison of the DGBRD values of β and Δtₙ (p=0.027 for the one-sided Wilcoxon signed-rank test). Comparing the overall DGBRD distributions between the biomarkers under two-sided Wilcoxon rank-sum testing only the DGBRD values of Pₙ and Δtₙ show significant differences for the periodic dose model (p=0.022). The corresponding one-sided tests identify a significantly larger DGBRD for ρₙ than for Δtₙ for both models (p=0.011 and p=0.026, respectively). In addition, no significant differences are identified between the DGBRD values obtained for each biomarker with both models by using either two-sided Wilcoxon rank-sum or signed-rank tests.

Based on this, it is possible to make patient-specific predictions of PSA based on mechanistic models to design personalized PSA monitoring strategies in accordance with aspects of the invention. The detection of a consistent rising trend in PSA constitutes a biochemical relapse, which is indicative of a potential PCa recurrence. Here, it is posited that patient-specific forecasts of PSA evolution obtained via mechanistic modeling of post-EBRT PSA dynamics can accurately identify relapsing patients earlier than the current PSA threshold criteria for detection (e.g., nadir+2 ng/ml). To this end, the previously proposed mechanistic models of PSA dynamics after EBRT in Lorenzo et al. (2019) are leveraged. These models feature key advantages for their clinical use to forecast PSA during post-EBRT patient monitoring. First, they are defined upon the essential mechanisms underlying the tumor response to radiation and the ensuing changes in PSA dynamics after EBRT conclusion (see STAR Methods and FIG. 1). Consequently, the models have a simple formulation with only four parameters to be identified patient-wise from the longitudinal PSA data that are routinely collected during standard patient follow-up (see STAR Methods and FIG. 1). Finally, the mathematical solutions to the model span the plateauing trend observed in non-relapsing patients as well as the biexponential response expected for relapsing patients.

In the investigations reported above, it is first demonstrated that the models exhibit a remarkable accuracy in reproducing the complete post-EBRT PSA dynamics observed in a new patient cohort from a different center to the one from the previous study Lorenzo et al (2019), thereby providing preliminary evidence of cross-institutional validation. It is further demonstrated that the models can provide reasonably accurate short-term forecasts of PSA for both non-relapsing and relapsing patients over the course of post-EBRT monitoring. To this end, the experiments focused on predicting the immediately next two PSA values that were not used to parameterize the models in the serial n_{P,fit} scenarios run for each patient (n_{P,fit}=5, . . . , n_{P-1}) during the fitting-forecasting study. As noted, the short-term PSA predictions correspond to a time horizon of approximately one year according to the PSA testing frequency in the cohort (see Table 1). This prediction time horizon overlaps with standard PSA monitoring protocols, which usually define routine PSA tests more frequently over the first years following EBRT termination (e.g., every 3 to 6 months) and more sparsely afterwards (e.g., every 6 to 12 months), unless the collected PSA values rise suspicion of a potential relapse that would warrant more frequent testing (e.g., moderately high PSA levels, an incipient rising trend). Thus, the model forecasts could facilitate the systematic design of personalized monitoring plans, which can be adapted as the collection of further PSA data become available to inform the models. For instance, the consistent prediction of a plateauing trend during post-EBRT monitoring could be used to extend the time interval between consecutive PSA tests. Conversely, the detection of rising PSA dynamics would motivate a decision to prescribe more frequent PSA tests to confirm a biochemical relapse and then proceed to assess PCa recurrence (e.g., via biopsy and medical imaging). Hence, the mechanistic modeling forecasts could advance patient monitoring after EBRT from routine and observational PSA testing to a dynamic predictive paradigm optimizing PSA data collection on a patient-specific basis.

The principles of the methods and systems disclosed herein provide promising model-based biomarkers to early detect biochemical relapse. In addition to the explicit forecast of PSA values with the models, we further explore the performance of model-based biomarkers of biochemical relapse. By analyzing the global model fits to the whole PSA dataset available for each patient, four candidate biomarkers of biochemical relapse are identified: the proliferation rate of tumor cells (ρₛ), its ratio to the radiation-induced tumor cell death rate (β), the PSA nadir (Pₙ), and the time to PSA nadir since EBRT termination (Δtₙ).

As in Lorenzo et al. (2019), a superior performance is observed in classifying relapsing patients for the biomarkers that are directly related to the underlying tumor dynamics (ρₛ and β) than for the biomarkers that are more closely linked to PSA dynamics (ρₙ and Δtₙ; see FIGS. 4A-4D and Table 2). In particular, the results show that relapsing patients exhibit high ρₛ and β (see FIGS. 3A through 3H). A high proliferation activity measured via Ki-67 staining in PCa tissue samples has been correlated with worse prognosis, radiotherapeutic outcome, and survival. An elevated tumor cell proliferation has also been correlated with an increased risk of PCa aggressiveness in terms of a higher Gleason score, which is a histopathological metric that is ubiquitously used in the clinical management of PCa. In particular, a higher pre-treatment Gleason score has been linked to higher probability of both local and distant PCa recurrence. The measurement of both the Ki-67 staining index and the Gleason score require an invasive approach to extract patient-specific tissue samples. However, the mechanistic models could provide a non-invasive surrogate method to estimate the proliferation activity of PCa in terms of ρₛ and β, and thereby refine the estimation of patient-specific prognosis to guide therapeutic planning.

Pₙ and Δtₙ are common metrics in clinical studies of PSA dynamics after EBRT. According to the modeling framework, a high PSA nadir or a short time to reach it after EBRT conclusion are predictive for biochemical relapse (see FIGS. 3A through 3H). Indeed, previous clinical studies have also linked these observations to worse prognosis, such as a higher likelihood of tumor recurrence and reduced patient survival. The estimation of Pₙ and Δtₙ with the models relies on ρₛ and β, but also on other model quantities that are not significantly different between non-relapsing and relapsing patients (see STAR Methods herein). This may partially explain their comparatively poorer performance in identifying biochemical relapse with respect to ρₛ and β. Additionally, the post-EBRT PSA doubling time during biochemical relapse, a PSA metric linked to a poor PCa prognosis, can also be estimated by leveraging the models in Lorenzo et al. (2019).

Therefore, it is believed that the model-based biomarkers could be leveraged to assess the probability of tumor recurrence and estimate patient survival after EBRT, thereby assisting the treating physician in therapeutic decision-making. Additionally, the performance of the model-based biomarkers to early identify biochemical relapse in the course of post-EBRT PSA monitoring was investigated. The results show that the estimation of ρₛ, β, ρₙ, and Δtₙ with a fraction of the total PSA values available for each patient also enables to identify relapsing patients (see FIGS. 6A-6D and Table 3). In general, a similar classifier performance as in the global fitting scenario (see FIGS. 4A-4D and Table 2) is observed, although a more conservative cutoff value may be required to optimally detect biochemical relapse with ρₛ (see FIGS. 6A-6D and Table 3). The promising early classifier performance reported herein suggests that the model-based biomarkers are robust with respect to the amount of data required to identify biochemical relapse, and thus may enable to anticipate the diagnosis of this event with respect to current methods relying on PSA threshold criteria (e.g., nadir+2 ng/ml). The preliminary investigation of this hypothesis reported herein reveals that ρₛ, β, and Pₙ may enable to detect biochemical relapse a median of 125 to 971.5 days (or equivalently 4.2 to 32.4 months), significantly outperforming the standard clinical practice. Interestingly, while Pₙ exhibits a poorer classifier performance than ρₛ and β according to the ROC curve analyses in this study (see Table 2 and Table 3), this biomarker rendered the earliest detection of biochemical relapse in the cohort.

Aspects of the methods and systems disclosed herein provide a flexible modeling framework to address practical and research applications. Regarding the comparative performance of the periodic and of the single dose model, a study leading to this disclosure does not provide conclusive results to select one over the other. First, both models show a remarkable agreement during fitting and forecasting PSA (e.g., see FIGS. 2A-2F and 5A-5I). Despite the significantly superior fitting and forecasting results obtained with the single dose model for each patient in the non-relapsing subgroup, this difference is marginal in absolute terms. This observation was also confirmed when a comparison of the overall distributions of fit quality and prediction metrics did not identify significant differences in neither patient subgroup in the global fitting and the fitting-forecasting studies. Additionally, no significant differences were found in the fitting and forecasting of PSA values in the relapsing subgroup. Second, both models provide the same biomarkers of biochemical relapse, which exhibit comparatively equivalent classifier performance and predictive ability. This is demonstrated in the ROC analysis of the global fitting and the fitting-forecasting results as well as in the analysis of the DGBRD metric (see FIGS. 4A-4D, 6A-6D, and 7 and Tables 2, 3, and 4). In the previous study Lorenzo et al. (2019), the single dose model exhibited an extraordinary performance despite its simplicity, which was observed again in the present work. Based on the similar performance of both models, it is believed that the simplicity of the single dose model makes it more amenable to facilitate the fitting and forecasting of PSA dynamics in real clinical scenarios. However, since the periodic dose model and the general parent formulation from which the models are derived account for the timing of radiation doses (see STAR methods herein), these modeling options may also be of interest to investigate the personalization of radiation plans (e.g., dose escalation, hypofractionation).

Aspects of the methods and systems disclosed herein can provide a robust clinical implementation. Around 20% to 50% of PCa patients undergoing radiotherapy are estimated to exhibit a biochemical relapse within 5 to 10 years of treatment conclusion. Their early identification and the accurate estimation of the severity of their tumor recurrence is crucial to optimize disease control and survival. Further developments of the patient-specific forecasting methods based on mechanistic models of PSA dynamics could constitute a robust enabling technology to accurately address those timely needs in post-treatment patient monitoring. In particular, casting the models in a Bayesian framework would advance the current state of the forecasting technology by incorporating the uncertainty in PSA values as well as the uncertainty emanating from the model parameterization and ensuing predictions. It is believed that this approach may be used to seamlessly integrate the mechanistic model predictions with uncertainty quantification and risk assessment. This strategy has the potential to guide clinical decision-making during the post-treatment monitoring of individual patients, including the frequency of PSA data collection, the timing of tests to ascertain the suspicion of tumor recurrence, the estimation of clinical risks and survival (e.g., biochemical relapse, local and distant recurrence, or death), and the planning of optimal primary and salvage treatments by maximizing therapeutic outcomes and minimizing toxicities.

Unlike the current conventional observational standard metrics used in clinics, a patient-specific biomechanistic model of prostate-specific antigen (PSA) dynamics after external radiotherapy according to some aspects of the methods and systems disclosed herein may help oncologists treating prostate cancer (for which PSA is a standard-of-care biomarker) who want to early identify tumor recurrence after external radiotherapy to anticipate secondary treatments and maximize the chances of tumor control by providing patient-specific forecasts of PSA dynamics and new patient-specific model-based biomarker values that identify the patient as relapsing versus non-relapsing and facilitating the early detection and treatment of prostate cancer recurrence.

As previously noted above, though the foregoing detailed description describes certain aspects of one or more particular embodiments of the invention, alternatives could be adopted by one skilled in the art. As such, and again as was previously noted, the invention is not necessarily limited to any particular embodiment described herein or illustrated in the drawings.

1. A method of predicting relapse of a prostate cancer patient treated by radiation therapy, the method comprising:
collecting patient-specific PSA data comprising nₚ measurements of serum Prostate Specific Antigen (PSA) at nₚ dates for a newly-diagnosed prostate cancer patient before and after an external beam radiation therapy (EBRT) regimen;
identifying patient-specific parameters P₀, ρₙ, ρₛ, ρ_{d}, and R_{D} that render an optimal fit of the patient-specific PSA data to a personalized model;
calculating, using the personalized model and the patient-specific parameters, a set of one or more predicted PSA values at one or more time horizons from 0 to 2 years after the last PSA measurement; and
predicting if and/or when a relapse of the prostate cancer patient will occur based on the one or more predicted PSA values.

2. The method of claim 1, wherein the personalized model has the equation
$$P ( t ) = \{ \begin{matrix}{P}_{0} {e}^{{ρ}_{n} t} & t < {t}_{D} \\ {P}_{0} {e}^{{t}_{D} ( {ρ}_{n} - {ρ}_{s} )} [ {R}_{D} {e}^{{ρ}_{s} t} + ( 1 - {R}_{D} ) {r}^{{t}_{D} ( {ρ}_{s} + {ρ}_{d} )} {e}^{- {ρ}_{d} t} ] & t > {t}_{D}\end{matrix}$$
where P(t) represents the serum PSA value at time t, time t=0 corresponds to the first available PSA measurement for the patient, time t=t_{D} corresponds to onset of radiotherapy; parameter P₀ represents the baseline PSA as measured prior to radiotherapy; parameter ρₙ represents the net proliferation rate of prostate cancer cells before radiotherapy, parameter ρₛ represents the net proliferation rate of the prostate cancer cells surviving the radiation treatment, parameter ρ_{d} represents the radiation-induced death rate of prostate cancer cells, and parameter R_{D} represents the fraction of prostate cancer cells that survive the radiation treatment.

3. The method of claim 1, the method further comprising
choosing a course of action in the post-treatment monitoring of a prostate cancer patient based on the prediction.

4. The method of claim 3, wherein the course of action includes at least one of frequency of future PSA tests and whether to investigate a potential tumor recurrence.

5. The method of claim 1, the method further comprising calculating a panel of model-based biomarkers of biochemical relapse, which allow early detection of a consistent rising trend in PSA dynamics after radiotherapy.

6. The method of claim 5, wherein the model-based biomarkers are proliferation rate of the surviving prostate cancer cells ρₛ, ratio of the proliferation rate of the surviving prostate cancer cells and the radiation-induced death rate of prostate cancer cells β=ρₛ/ρ_{d}, PSA nadir Pₙ, and time to PSA nadir after termination of radiotherapy Δtₙ.

7. The method of claim 6, the method further comprising:
making a comparison of the patient-specific values of the model-based biomarkers to corresponding population-based thresholds; and
classifying the patient as relapsing or non-relapsing based on the comparison.

8. The method of claim 1, the method further comprising:
updating the patient-specific parameters and ensuing predictions by repeating the steps of collecting patient-specific PSA data, identifying the patient-specific parameters, calculating one or more predicted PSA values, and making a prediction.

9. The method of claim 1, cast in a Bayesian framework,
wherein prior distributions of the patient-specific parameters are determined from fitting of the personalized model to all available PSA data from each patient in a population,
wherein prior distributions of measurement errors are estimated from previous studies, and
wherein Bayesian inference is used to determine posterior distributions of model parameters, model predictions of PSA, and probabilistic risk of biochemical relapse according to model-based biomarkers of biochemical relapse.

10. The method of claim 1, the method further comprising:
using the predicted PSA values to determine an optimal frequency of future PSA tests for the patient.

11. The method of claim 10, wherein the optimal frequency of future PSA tests is selected such that the number of future tests is minimal.

12. The method of claim 10, wherein the optimal frequency of future PSA tests is selected such that accuracy of the model predictions is maximal.

13. The method of claim 10, wherein the optimal frequency of future PSA tests is selected such that the probabilistic risk of late detection of biochemical relapse is minimal.

14. The method of claim 1, wherein the personalized model is obtained using only PSA values collected before the EBRT regimen, and further comprising:
optimizing a radiotherapeutic plan for the patient, including total dose and onset date of the EBRT regimen, using values of the patient-specific parameters such that the radiotherapeutic plan minimizes the PSA after radiotherapy to a flat trend having constant value and using a minimal radiation dose.

15. The method of claim 14, wherein minimizing the PSA after radiotherapy to a flat trend having constant value and using a minimal radiation dose is cast in a probabilistic formulation configured to minimize probabilistic risk of biochemical relapse after the EBRT regimen using the minimal radiation dose.

16. The method of claim 14, wherein the personalized model has the equation
P(t)=P₀θ₁[R_{d}ⁿ^{_{d}}e^{ρ}^{ₛ}^{t}+(1−R_{d})(Σᵢ₌₁ⁿ^{_{d}}R_{d}ⁱ⁻¹e^{(t}^{ᵢ}^{-t}^{₁}^{)(ρ}^{ₛ}^{+ρ}^{_{d}}^{))θ}₂e^{-ρ}^{_{d}}^{t}],t>tₙ_{_{d}}.

17. The method of claim 1, wherein the personalized model comprises an additional positive exponential rate representing increase in benign prostatic hyperplasia (BPH) caused by coexisting BPH.

18. The method of claim 1, wherein the predicted PSA value is a nadir of a plurality of PSA values obtained as a function of the personalized model and the patient-specific parameters during the time horizon.

19. A software product comprising:
a set of instructions on a non-transitory medium configured to cause a computer system to implement the identifying step and the calculating step of claim 1.

20. The software product of claim 19, wherein the set of instructions is configured to cause the computer to predict if and/or when a relapse of the prostate cancer patient will occur based on the one or more predicted PSA values.

21. A system for predicting relapse of a prostate cancer patient treated by radiation therapy, the system comprising:
a computer system comprising at least one processor configured to run the software product of claim 19; and
a user interface configured to provide to a user the predicted PSA values and/or whether and/or when a relapse is predicted to occur.
